SEQUENCE LISTING (56 sequences)

SEQ ID NO: 1 - DNA, 3912 bp, Saccharomyces cerevisiae
  source 1..3912: mol_type = genomic DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggttga

SEQ ID NO: 2 - protein, 1303 aa, Saccharomyces cerevisiae
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGG

SEQ ID NO: 3 - DNA, 4767 bp, Saccharomyces cerevisiae
  source 1..4767: mol_type = genomic DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 4 - protein, 1588 aa, Saccharomyces cerevisiae
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 5 - DNA, 4767 bp, synthetic construct
  misc_feature 1..4767: note = Synthetic nucleic acid
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccccatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 6 - protein, 1588 aa, synthetic construct
  REGION 1..1588: note = Synthethic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAPYKPSVTPVMTISQDKYQWHVVPGSQMLVHQGVAQFEKWTGFKGPFKAIFDAVTKE

SEQ ID NO: 7 - DNA, 4767 bp, synthetic construct
  misc_feature 1..4767: note = Synthetic nucleic acid
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaaaagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 8 - protein, 1588 aa, synthetic construct
  REGION 1..1588: note = Synthetic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVKADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 9 - DNA, 4767 bp, synthetic construct
  misc_feature 1..4767: note = Synthetic nucleic acid
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaactggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 10 - protein, 1588 aa, synthetic construct
  REGION 1..1588: note = Synthetic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVIGAVNTVIPLGNKKFKGDNTDWLGIRNALINNGVPEYVGHTAGLVIGAGGTSRAALYALHSLGCKKIFIINWTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 11 - DNA, 4767 bp, synthetic construct
  misc_feature 1..4767: note = Synthetic nucleic acid
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattgggaaaagaaaaacttttggacggaaacaagaactttggcggtgctgcagtcacaattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 12 - protein, 1588 aa, synthetic construct
  REGION 1..1588: note = Synthetic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLGKEKLLDGNKNFGGAAVTIPLKLDIMQYMDELTDAAKVIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 13 - DNA, 4767 bp, synthetic construct
  misc_feature 1..4767: note = Synthetic nucleic acid
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcaggattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 14 - protein, 1588 aa, synthetic construct
  REGION 1..1588: note = Synthetic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVRIPLKLDIMQYMDELTDAAKVIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 15 - DNA, 4767 bp, synthetic construct
  misc_feature 1..4767: note = Synthetic nucleic acid
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttcatggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 16 - protein, 1588 aa, synthetic construct
  REGION 1..1588: note = Synthetic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVHGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 17 - DNA, 4767 bp, synthetic construct
  misc_feature 1..4767: note = Synthetic nucleic acid
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaaggttacaggattcaagggccctttcaaggccatttttgatgccgttacgaaagagtag

SEQ ID NO: 18 - protein, 1588 aa, synthetic construct
  REGION 1..1588: note = Synthetic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKVTGFKGPFKAIFDAVTKE

SEQ ID NO: 19 - DNA, 4767 bp, synthetic construct
  misc_feature 1..4767: note = Synthetic nucleic acid
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttattggtgctgtaaacaaagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 20 - protein, 1588 aa, synthetic construct
  REGION 1..1588: note = Synthetic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVIGAVNKVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 21 - DNA, 4767 bp, synthetic construct
  misc_feature 1..4767: note = Synthetic nucleic acid
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgttattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccgccctttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 22 - protein, 1588 aa, synthetic construct
  REGION 1..1588: note = Synthetic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLLLDIMQYMDELTDAAKVIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 23 - DNA, 4767 bp, synthetic construct
  misc_feature 1..4767: note = Synthetic nucleic acid
  source 1..4767: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccactctagatcgccaattttacataacactggctatgaaattttaggtttacctcacaagttcgataaatttgaaactgaatccgcacaattggtgaaagaaaaacttttggacggaaacaagaactttggcggtgctgcagtcacaattcctctgaaattagatataatgcagtacatggatgaattgactgatgctgctaaagttattggtgctgtaaacacagttataccattgggtaacaagaagtttaagggtgataataccgactggttaggtatccgtaatgccttaattaacaatggcgttcccgaatatgttggtcataccgctggtttggttatcggtgcaggtggcacttctagagccccactttacgccttgcacagtttaggttgcaaaaagatcttcataatcaacaggacaacttcgaaattgaagccattaatagagtcacttccatctgaattcaacattattggaatagagtccactaaatctatagaagagattaaggaacacgttggcgttgctgtcagctgtgtaccagccgacaaaccattagatgacgaacttttaagtaagctggagagattccttgtgaaaggtgcccatgctgcttttgtaccaaccttattggaagccgcatacaaaccaagcgttactcccgttatgacaatttcacaagacaaatatcaatggcacgttgtccctggatcacaaatgttagtacaccaaggtgtagctcagtttgaaaagtggacaggattcaagggccctttcaaggccatttttgatgccgttacgaaagagtag

SEQ ID NO: 24 - protein, 1588 aa, synthetic construct
  REGION 1..1588: note = Synthetic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVIGAVNTVIPLGNKKFKGDNTDWLGIRNALINNGVPEYVGHTAGLVIGAGGTSRAPLYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 25 - DNA, 5064 bp, synthetic construct
  misc_feature 1..5064: note = Synthetic nucleic acid
  source 1..5064: mol_type = other DNA
atggtgcagttagccaaagtcccaattctaggaaatgatattatccacgttgggtataacattcatgaccatttggttgaaaccataattaaacattgtccttcttcgacatacgttatttgcaatgatacgaacttgagtaaagttccatactaccagcaattagtcctggaattcaaggcttctttgccagaaggctctcgtttacttacttatgttgttaaaccaggtgagacaagtaaaagtagagaaaccaaagcgcagctagaagattatcttttagtggaaggatgtactcgtgatacggttatggtagcgatcggtggtggtgttattggtgacatgattgggttcgttgcatctacatttatgagaggtgttcgtgttgtccaagtaccaacatccttattggcaatggtcgattcctccattggtggtaaaactgctattgacactcctctaggtaaaaactttattggtgcattttggcaaccaaaatttgtccttgtagatattaaatggctagaaacgttagccaagagagagtttatcaatgggatggcagaagttatcaagactgcttgtatttggaacgctgacgaatttactagattagaatcaaacgcttcgttgttcttaaatgttgttaatggggcaaaaaatgtcaaggttaccaatcaattgacaaacgagattgacgagatatcgaatacagatattgaagctatgttggatcatacatataagttagttcttgagagtattaaggtcaaagcggaagttgtctcttcggatgaacgtgaatccagtctaagaaaccttttgaacttcggacattctattggtcatgcttatgaagctatactaaccccacaagcattacatggtgaatgtgtgtccattggtatggttaaagaggcggaattatcccgttatttcggtattctctcccctacccaagttgcacgtctatccaagattttggttgcctacgggttgcctgtttcgcctgatgagaaatggtttaaagagctaaccttacataagaaaacaccattggatatcttattgaagaaaatgagtattgacaagaaaaacgagggttccaaaaagaaggtggtcattttagaaagtattggtaagtgctatggtgactccgctcaatttgttagcgatgaagacctgagatttattctaacagatgaaaccctcgtttaccccttcaaggacatccctgctgatcaacagaaagttgttatcccccctggttctaagtccatctccaatcgtgctttaattcttgctgccctcggtgaaggtcaatgtaaaatcaagaacttattacattctgatgatactaaacatatgttaaccgctgttcatgaattgaaaggtgctacgatatcatgggaagataatggtgagacggtagtggtggaaggacatggtggttccacattgtcagcttgtgctgaccccttatatctaggtaatgcaggtactgcatctagatttttgacttccttggctgccttggtcaattctacttcaagccaaaagtatatcgttttaactggtaacgcaagaatgcaacaaagaccaattgctcctttggtcgattctttgcgtgctaatggtactaaaattgagtacttgaataatgaaggttccctgccaatcaaagtttatactgattcggtattcaaaggtggtagaattgaattagctgctacagtttcttctcagtacgtatcctctatcttgatgtgtgccccatacgctgaagaacctgtaactttggctcttgttggtggtaagccaatctctaaattgtacgtcgatatgacaataaaaatgatggaaaaattcggtatcaatgttgaaacttctactacagaaccttacacttattatattccaaagggacattatattaacccatcagaatacgtcattgaaagtgatgcctcaagtgctacatacccattggccttcgccgcaatgactggtactaccgtaacggttccaaacattggttttgagtcgttacaaggtgatgccagatttgcaagagatgtcttgaaacctatgggttgtaaaataactcaaacggcaacttcaactactgtttcgggtcctcctgtaggtactttaaagccattaaaacatgttgatatggagccaatgactgatgcgttcttaactgcatgtgttgttgccgctatttcgcacgacagtgatccaaattctgcaaatacaaccaccattgaaggtattgcaaaccagcgtgtcaaagagtgtaacagaattttggccatggctacagagctcgccaaatttggcgtcaaaactacagaattaccagatggtattcaagtccatggtttaaactcgataaaagatttgaaggttccttccgactcttctggacctgtcggtgtatgcacatatgatgatcatcgtgtggccatgagtttctcgcttcttgcaggaatggtaaattctcaaaatgaacgtgacgaagttgctaatcctgtaagaatacttgaaagacattgtactggtaaaacctggcctggctggtgggatgtgttacattccgaactaggtgccaaattagatggtgcagaacctttagagtgcacatccaaaaagaactcaaagaaaagcgttgtcattattggcatgagagcagctggcaaaactactataagtaaatggtgcgcatccgctctgggttacaaattagttgacctagacgagctgtttgagcaacagcataacaatcaaagtgttaaacaatttgttgtggagaacggttgggagaagttccgtgaggaagaaacaagaattttcaaggaagttattcaaaattacggcgatgatggatatgttttctcaacaggtggcggtattgttgaaagcgctgagtctagaaaagccttaaaagattttgcctcatcaggtggatacgttttacacttacatagggatattgaggagacaattgtctttttacaaagtgatccttcaagacctgcctatgtggaagaaattcgtgaagtttggaacagaagggaggggtggtataaagaatgctcaaatttctctttctttgctcctcattgctccgcagaagctgagttccaagctctaagaagatcgtttagtaagtacattgcaaccattacaggtgtcagagaaatagaaattccaagcggaagatctgcctttgtgtgtttaacctttgatgacttaactgaacaaactgagaatttgactccaatctgttatggttgtgaggctgtagaggtcagagtagaccatttggctaattactctgctgatttcgtgagtaaacagttatctatattgcgtaaagccactgacagtattcctatcatttttactgtgcgaaccatgaagcaaggtggcaactttcctgatgaagagttcaaaaccttgagagagctatacgatattgccttgaagaatggtgttgaattccttgacttagaactaactttacctactgatatccaatatgaggttattaacaaaaggggcaacaccaagatcattggttcccatcatgacttccaaggattatactcctgggacgacgctgaatgggaaaacagattcaatcaagcgttaactcttgatgtggatgttgtaaaatttgtgggtacggctgttaatttcgaagataatttgagactggaacactttagggatacacacaagaataagcctttaattgcagttaatatgacttctaaaggtagcatttctcgtgttttgaataatgttttaacacctgtgacatcagatttattgcctaactccgctgcccctggccaattgacagtagcacaaattaacaagatgtatacatctatgggaggtatcgagcctaaggaactgtttgttgttggaaagccaattggccccgggaaaatgccttccaaactcgccatcacttccatgtcacttggccggtgttatgccggccactccttcaccactaagctcgatatggcccggaaatatggctatcaaggcctagagctcttccacgaggacttggctgatgtagcctatcgtctctccggagagaccccttccccatgtggcccgtccccagcagcccagctctcggctgcccgtcaaatcctccgcatgtgccaagtcagaaacattgaaatcgtctgcctccagcccttcagccagtacgacggcctactcgaccgcgaggagcacgagcgccgtctggagcagctcgagttctggatcgagctcgcccacgagcttgacacagacattatccaaatccccgccaactttctccccgccgaggaagtaactgaggacatttcgctcatcgtctcggaccttcaagaagtggccgacatgggcctgcaggccaacccacccatccgctttgtctacgaggctctgtgctggagcactcgtgtcgacacttgggagcgtagctgggaggtggtgcagagggtgaacaggcccaactttggcgtgtgcctggacactttcaacattgcggggcgggtatatgctgatccgacggttgcctctggccgcacccccaacgcggaggaagcgatacggaagtcgattgcgcgtctcgttgaaagggtcgatgtcagcaaggtcttttatgtgcaggttgtggacgctgagaagttgaagaagccgctggtgccgggtcatcggttttatgacccggagcagccggcgaggatgagctggtcaaggaactgcaggttattctacggggagaaggacagaggggcgtatttgcccgtcaaggagattgcctgggccttcttcaacgggctcggattcgagggttgggtcagtctggagctcttcaacagaagaatgtcggacacaggctttggggtgcccgaggagctggccaggagaggggccgtgtcgtgggcaaagctggtgagggacatgaagatcactgttgattcaccaacacaacaacaagccacacagcagcccatcaggatgctgtcgctgtcagcggctttgtaa

SEQ ID NO: 26 - protein, 1687 aa, synthetic construct
  REGION 1..1687: note = Synthetic polypeptide
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGPGKMPSKLAITSMSLGRCYAGHSFTTKLDMARKYGYQGLELFHEDLADVAYRLSGETPSPCGPSPAAQLSAARQILRMCQVRNIEIVCLQPFSQYDGLLDREEHERRLEQLEFWIELAHELDTDIIQIPANFLPAEEVTEDISLIVSDLQEVADMGLQANPPIRFVYEALCWSTRVDTWERSWEVVQRVNRPNFGVCLDTFNIAGRVYADPTVASGRTPNAEEAIRKSIARLVERVDVSKVFYVQVVDAEKLKKPLVPGHRFYDPEQPARMSWSRNCRLFYGEKDRGAYLPVKEIAWAFFNGLGFEGWVSLELFNRRMSDTGFGVPEELARRGAVSWAKLVRDMKITVDSPTQQQATQQPIRMLSLSAAL

SEQ ID NO: 27 - protein, 221 aa, Homo sapiens
MGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 28 - DNA, 40 bp, synthetic construct
  misc_feature 1..40: note = Synthetic oligonucleotide
  source 1..40: mol_type = other DNA
cgtagcatgcagtctagaaaaatgggtgacactaaggagc

SEQ ID NO: 29 - DNA, 45 bp, synthetic construct
  misc_feature 1..45: note = Synthetic oligonucleotide
  source 1..45: mol_type = other DNA
gacgacgttagtgacagaattcttatggaccagcttcagaacctg

SEQ ID NO: 30 - DNA, 25 bp, synthetic construct
  misc_feature 1..25: note = Synthetic oligonucleotide
  source 1..25: mol_type = other DNA
cgtagcatgcagtctagaaaaatgg

SEQ ID NO: 31 - DNA, 22 bp, synthetic construct
  misc_feature 1..22: note = Synthetic oligonucleotide
  source 1..22: mol_type = other DNA
gacgacgttagtgacagaattc

SEQ ID NO: 32 - DNA, 45 bp, synthetic construct
  misc_feature 1..45: note = Synthetic oligonucleotide
  misc_difference 30: note = n is a, c, g, or t
  source 1..45: mol_type = other DNA
ctattgacacttattgtgagcaaaaggagnrkgctatgaacgttg

SEQ ID NO: 33 - DNA, 45 bp, synthetic construct
  misc_feature 1..45: note = Synthetic oligonucleotide
  misc_difference 30: note = n is a, c, g, or t
  source 1..45: mol_type = other DNA
ctattgacacttattgtgagcaaaaggagnykgctatgaacgttg

SEQ ID NO: 34 - DNA, 29 bp, synthetic construct
  misc_feature 1..29: note = Synthetic oligonucleotide
  source 1..29: mol_type = other DNA
ctccttttgctcacaataagtgtcaatag

SEQ ID NO: 35 - DNA, 45 bp, synthetic construct
  misc_feature 1..45: note = Synthetic oligonucleotide
  misc_difference 31: note = n is a, c, g, or t
  source 1..45: mol_type = other DNA
gacacttattgtgagcaaaaggagtgggctnrkaacgttggtgac

SEQ ID NO: 36 - DNA, 43 bp, synthetic construct
  misc_feature 1..43: note = Synthetic oligonucleotide
  misc_difference 29: note = n is a, c, g, or t
  source 1..43: mol_type = other DNA
cacttattgtgagcaaaaggagtgggctnykaacgttggtgac

SEQ ID NO: 37 - DNA, 24 bp, synthetic construct
  misc_feature 1..24: note = Synthetic oligonucleotide
  source 1..24: mol_type = other DNA
ctccttttgctcacaataagtgtc

SEQ ID NO: 38 - DNA, 46 bp, synthetic construct
  misc_feature 1..46: note = Synthetic oligonucleotide
  misc_difference 27: note = n is a, c, g, or t
  source 1..46: mol_type = other DNA
ctttggacatggttttcttggaccatnrkaaggacagatatttgcc

SEQ ID NO: 39 - DNA, 46 bp, synthetic construct
  misc_feature 1..46: note = Synthetic oligonucleotide
  misc_difference 27: note = n is a, c, g, or t
  source 1..46: mol_type = other DNA
ctttggacatggttttcttggaccatnykaaggacagatatttgcc

SEQ ID NO: 40 - DNA, 26 bp, synthetic construct
  misc_feature 1..26: note = Synthetic oligonucleotide
  source 1..26: mol_type = other DNA
atggtccaagaaaaccatgtccaaag

SEQ ID NO: 41 - DNA, 53 bp, synthetic construct
  misc_feature 1..53: note = Synthetic oligonucleotide
  misc_difference 30: note = n is a, c, g, or t
  source 1..53: mol_type = other DNA
gtactgttttgttagctgacaacgttattnrkccaggtgctccagacttcttg

SEQ ID NO: 42 - DNA, 53 bp, synthetic construct
  misc_feature 1..53: note = Synthetic oligonucleotide
  misc_difference 30: note = n is a, c, g, or t
  source 1..53: mol_type = other DNA
gtactgttttgttagctgacaacgttattnykccaggtgctccagacttcttg

SEQ ID NO: 43 - DNA, 29 bp, synthetic construct
  misc_feature 1..29: note = Synthetic oligonucleotide
  source 1..29: mol_type = other DNA
aataacgttgtcagctaacaaaacagtac

SEQ ID NO: 44 - DNA, 47 bp, synthetic construct
  misc_feature 1..47: note = Synthetic oligonucleotide
  misc_difference 30: note = n is a, c, g, or t
  source 1..47: mol_type = other DNA
ctgttttgttagctgacaacgttatttgtnrkggtgctccagacttc

SEQ ID NO: 45 - DNA, 47 bp, synthetic construct
  misc_feature 1..47: note = Synthetic oligonucleotide
  misc_difference 30: note = n is a, c, g, or t
  source 1..47: mol_type = other DNA
ctgttttgttagctgacaacgttatttgtnykggtgctccagacttc

SEQ ID NO: 46 - DNA, 29 bp, synthetic construct
  misc_feature 1..29: note = Synthetic oligonucleotide
  source 1..29: mol_type = other DNA
acaaataacgttgtcagctaacaaaacag

SEQ ID NO: 47 - DNA, 103 bp, synthetic construct
  misc_feature 1..103: note = Synthetic oligonucleotide
  misc_difference 86: note = n is a, c, g, or t
  source 1..103: mol_type = other DNA
atttagaattcttatggaccagcttcagaacctggacccttatatatagccttctccaaaccgtcaacaacctctctatattcmyngaaagattgataatgag

SEQ ID NO: 48 - DNA, 102 bp, synthetic construct
  misc_feature 1..102: note = Synthetic oligonucleotide
  misc_difference 85: note = n is a, c, g, or t
  source 1..102: mol_type = other DNA
atttagaattctatggaccagcttcagaacctggacccttatatatagccttctccaaaccgtcaacaacctctctatattcmrngaaagattgataatgag

SEQ ID NO: 49 - DNA, 101 bp, synthetic construct
  misc_feature 1..101: note = Synthetic oligonucleotide
  misc_difference 83: note = n is a, c, g, or t
  source 1..101: mol_type = other DNA
atttagaattcttatggaccagcttcagaacctggacccttatatatagccttctccaaaccgtcaacaacctctctatamyncaagaaagattgataatg

SEQ ID NO: 50 - DNA, 101 bp, synthetic construct
  misc_feature 1..101: note = Synthetic oligonucleotide
  misc_difference 83: note = n is a, c, g, or t
  source 1..101: mol_type = other DNA
atttagaattcttatggaccagcttcagaacctggacccttatatatagccttctccaaaccgtcaacaacctctctatamrncaagaaagattgataatg

SEQ ID NO: 51 - DNA, 95 bp, synthetic construct
  misc_feature 1..95: note = Synthetic oligonucleotide
  misc_difference 77: note = n is a, c, g, or t
  source 1..95: mol_type = other DNA
atttagaattcttatggaccagcttcagaacctggacccttatatatagccttctccaaaccgtcaacaacctcmynatattccaagaaagattg

SEQ ID NO: 52 - DNA, 95 bp, synthetic construct
  misc_feature 1..95: note = Synthetic oligonucleotide
  misc_difference 77: note = n is a, c, g, or t
  source 1..95: mol_type = other DNA
atttagaattcttatggaccagcttcagaacctggacccttatatatagccttctccaaaccgtcaacaacctcmrnatattccaagaaagattg

SEQ ID NO: 53 - protein, 363 aa, Arabidopsis thaliana
MGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAALCLMNQDKVLMESWYHLKDAILDGGIPFNKAYGMSAFEYHGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKWICHDWSDEHCVKFLKNCYESLPEDGKVILAECILPETPDSSLSTKQVVHVDCIMLAHNPGGKERTEKEFEALAKASGFKGIKVVCDAFGVNLIELLKKL

SEQ ID NO: 54 - protein, 365 aa, Fragaria x ananassa
  source 1..365: note = Fragaria x ananassa is strawberry
MGSTGETQMTPTHVSDEEANLFAMQLASASVLPMVLKAAIELDLLEIMAKAGPGSFLSPSDLASQLPTKNPEAPVMLDRMLRLLASYSILTCSLRTLPDGKVERLYCLGPVCKFLTKNEDGVSIAALCLMNQDKVLVESWYHLKDAVLDGGIPFNKAYGMTAFDYHGTDPRFNKVFNKGMADHSTITMKKILETYKGFEGLKSIVDVGGGTGAVVNMIVSKYPSIKGINFDLPHVIEDAPQYPGVQHVGGDMFVSVPKGNAIFMKWICHDWSDEHCIKFLKNCYAALPDDGKVILAECILPVAPDTSLATKGVVHMDVIMLAHNPGGKERTEQEFEALAKGSGFQGIRVCCDAFNTYVIEFLKKI

SEQ ID NO: 55 - protein, 271 aa, Homo sapiens
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 56 - protein, 263 aa, Vanilla planifolia
MATTVATATRATENKTQTEENSQNGGQQTGHQEIGHKSLLKSDALYQYILETSVYPREPECLKELREITAKHPWNLMTTSADEGQFLGMLLKLINAKNTMEIGVFTGYSLLATALALPDDGKILAMDINRENYELGLPLIQKAGVAHKIDFREGPALPVLDELMKDESKHGSFDFIFVDADKDNYLNYHQRIIDLVKVGGVIGYDNTLWNGAVVLPPDAPMRKYIRYYRDFVIELNKELAADPRIEICQLPVGDGITLCRRVK

(19) United States
(12) Patent Application Publication
(10) Pub. No.: US 2026/0234676 A1
(43) Pub. Date: Aug. 13, 2026

(54) COMPOSITIONS AND METHODS FOR THE BIOSYNTHESIS OF VANILLIN OR VANILLIN BETA-D-GLUCOSIDE

(71) Applicants: International Flavors & Fragrances, Inc., New York, NY (US); Danstar Ferment AG, Zug (CH)

(72) Inventors: Joergen HANSEN, Frederiksberg (DK); Esben Halkjaer HANSEN, Frederiksberg (DK); Honey POLUR, Durham, NC (US); Joseph M. SHERIDAN, Royston (GB); Jonathan R. HEAL, Attenbrough (GB); William D.O. HAMILTON, Royston (GB)

(21) Appl. No.: 19/381,062

(22) Filed: Nov. 6, 2025

Related U.S. Application Data
(63) Continuation of application No. 19/175,009, filed on Apr. 10, 2025, now abandoned.
Application No. 19/175,009 is a continuation of application No. 18/826,474, filed on Sep. 6, 2024, now abandoned.
Application No. 18/826,474 is a continuation of application No. 18/403,926, filed on Jan. 4, 2024, now abandoned.
Application No. 18/403,926 is a continuation of application No. 18/342,129, filed on Jun. 27, 2023, now abandoned.
Application No. 18/342,129 is a continuation of application No. 17/230,533, filed on Apr. 14, 2021, now abandoned.
Application No. 17/230,533 is a division of application No. 16/243,670, filed on Jan. 9, 2019, now Pat. No. 11,008,592.
Application No. 16/243,670 is a division of application No. 14/236,991, filed on Aug. 7, 2012, now Pat. No. 10,208,293.
(60) Provisional application No. 61/521,090, filed on Aug. 8, 2011.
(60) Provisional application No. 61/522,096, filed on Aug. 10, 2011.

(51) Int. Cl. C12P 7/24; A23L 33/105; C07K 14/395; C12N 9/04; C12N 9/10; C12N 9/88; C12N 15/82; C12P 19/46

(52) U.S. Cl. CPC C12P 7/24; A23L 33/105; C12N 9/0006; C12N 9/1011; C12N 9/88; C12P 19/46; C12Y 201/01006; C12Y 402/01118; C07K 14/395; C12N 15/8221; C12Y 101/01025

(57) ABSTRACT

Recombinant microorganisms, plants, and plant cells are disclosed that have been engineered to express a mutant AROM polypeptide and/or mutant catechol-O-methyltransferase polypeptide alone or in combination with one or more vanillin biosynthetic enzymes or UDP-glycosyltransferases (UGTs). Such microorganisms, plants, or plant cells can produce vanillin or vanillin beta-D-glucoside.

### CROSS-REFERENCE TO RELATED APPLICATIONS

This application is a continuation of U.S. Ser. No. 19/175,009, filed, Apr. 10, 2025, now abandoned, which is a continuation of U.S. Ser. No. 18/826,474, filed Sep. 6, 2024, now abandoned, which is a continuation of U.S. Ser. No. 18/403,926, filed Jan. 4, 2024, now abandoned, which is a continuation of U.S. Ser. No. 18/342,129, filed Jun. 27, 2023, now abandoned, which is a continuation of U.S. Ser. No. 17/230,533, filed Apr. 14, 2021, now abandoned, which is a divisional of U.S. Ser. No. 16/243,670 filed Jan. 9, 2019, now U.S. Pat. No. 11,008,592, issued May 18, 2021, which is a divisional of U.S. Ser. No. 14/236,991 filed Apr. 7, 2014, now U.S. Pat. No. 10,208,293, issued on Feb. 19, 2019, which is the National Stage of International Application No. PCT/US2012/049842 filed Aug. 7, 2012, which claims the benefit of priority of U.S. Provisional Application Nos. 61/521,090, filed Aug. 8, 2011 and 61/522,096, filed Aug. 10, 2011, the teachings of which are incorporated herein by reference in their entireties.

### INTRODUCTION

### Reference to an Electronic Sequence Listing

This application contains a Sequence Listing, which has been submitted electronically in xml format and is hereby incorporated by reference in its entirety. Said xml copy, created on May 6, 2026, is named SeqList3-389234-46002.xml and is 158,586 bytes in size.

### BACKGROUND OF THE INVENTION

Vanillin is one of the world's most important flavor compounds with a global market of 180 million dollars. Natural vanillin is derived from the cured seed pods of the vanilla orchid (Vanilla planifolia), but most of the world's vanillin is synthesized from petrochemicals or wood pulp lignins. Production of natural vanillin from the vanilla pod is a laborious and slow process, which requires hand pollination of the flowers and a 1-6 month curing process of the harvested green vanilla pods (Ramachandra & Ravishankar (2000) J Sci. Food Agric. 80:289-304). Production of 1 kilogram (kg) of vanillin requires approximately 500 kg of vanilla pods, corresponding to pollination of approximately 40,000 flowers. Today only about 0.25% (40 tons out of 16,000) of vanillin sold annually originates from vanilla pods, while most of the remainder is synthesized chemically from lignin or fossil hydrocarbons, in particular guaiacol. Synthetically produced vanillin is sold for approximately $15 per kg, compared to prices of $1200-4000 per kg for natural vanillin (Walton, et al. (2003) Phytochemistry 63:505-515).

### SUMMARY OF THE INVENTION

This invention provides a method for producing vanillin and/or vanillin beta-D-glucoside. The method of the invention involves the steps of (a) providing a recombinant host capable of producing vanillin, wherein said recombinant host harbors a heterologous nucleic acid encoding a mutant Arom Multifunctional Enzyme (AROM) polypeptide and/or a mutant Catechol-O-Methyl Transferase (COMT) polypeptide; (b) cultivating said recombinant host for a time sufficient for said recombinant host to produce vanillin and/or vanillin glucoside; and (c) isolating vanillin and/or vanillin glucoside from said recombinant host or from the cultivation supernatant, thereby producing vanillin and/or vanillin beta-D-glucoside.

In one embodiment, a mutant AROM polypeptide is provided, wherein said mutant has decreased shikimate dehydrogenase activity relative to a corresponding AROM polypeptide that lacks the mutation. In accordance with this embodiment, the mutant AROM polypeptide can have one or more mutations in domain 5, a deletion of at least portion of domain 5, or lack domain 5.

In another embodiment, the mutant AROM polypeptide is a fusion polypeptide that includes (i) an AROM polypeptide described herein, e.g., an AROM polypeptide comprising a deletion of at least a portion of domain 5 or an AROM polypeptide lacking domain 5; and (ii) a polypeptide having 3-dehydroshikimate dehydratase (3DSD) activity.

In a further embodiment, a mutant COMT polypeptide is provided, wherein said mutant has one or more improved properties. In particular, the invention provides mutant COMT polypeptides that preferentially catalyze methylation at the meta position of protocatechuic acid, and/or protocatechuic aldehyde, and/or protocatechuic alcohol rather than at the para position.

Any of the polypeptides described herein further can include a purification tag, a chloroplast transit peptide, a mitochondrial transit peptide, an amyloplast peptide, signal peptide, or a secretion tag at the N or C-terminus of the polypeptide.

In some embodiments, the host can be a microorganism, e.g., a yeast such as Saccharomyces cerevisiae, Schizosaccharomyces pombe or Escherichia coli. Alternatively, the host can be a plant or plant cell (e.g., a Physcomitrella or tobacco plant or plant cell).

Any of the hosts described herein further can include a gene encoding an aromatic carboxylic acid reductase (ACAR); a 3-dehydroshikimate dehydratase (3DSD); a uridine 5′-diphosphoglucosyl transferase (UGT); a phosphopantetheine transferase (PPTase); a wild-type AROM; and/or a wild type O-methyltransferase (OMT).

An isolated nucleic acid encoding a mutant COMT polypeptide or mutant AROM polypeptide is also provided.

### BRIEF DESCRIPTION OF THE DRAWINGS

FIG. 1 is a schematic of de novo biosynthesis of vanillin (4) and outline of the different vanillin catabolites and metabolic side products, i.e., dehydroshikimic acid (1), protocatechuic acid (2), protocatechuic acid aldehyde (3), vanillic acid (5), protocatechuic alcohol (6), vanillyl alcohol (7), and vanillin β-D-glucoside (8), found in an organism expressing 3DSD, ACAR, OMT, and UGT and a phophopantheteine transferase (PPTase). Open arrows show primary metabolic reactions in yeast; black arrows show enzyme reactions introduced by metabolic engineering; diagonally striped arrows show undesired inherent yeast metabolic reactions.

FIG. 2 is a schematic depiction of the cellular biosynthesis of the aromatic amino acids tyrosine, tryptophan and phenylalanine using a wild-type AROM polypeptide (left) and a mutant AROM polypeptide lacking domain 5 (right).

FIG. 3A shows production of vanillin (VG) and iso-vanillin (IsoVg) in yeast strain expressing various mutant COMT polypeptides.

FIG. 3B and FIG. 3C show production of vanillin (VG) and iso-vanillin (IsoVg) by COMT polypeptides isolated from various sources as compared to human OMT (Hs-OMT) and the human L198Y OMT mutant (HS-OMT L198Y). Phytophthera infestans (PI), Catharanthus roseus (CR), Yarrowia lipolytica (YL), Ciona intestinalis GENBANK Accession Nos. XP_002121420 and XP_002131313 (CI-1 and CI-2), Capsasproa owczarzaki (CO), Chaetomium thermophilum (CT), Clavispora lusitaniae (CL), Paracoccidioides sp. ‘lutzii’ Pb01 (PL), Vanilla planifolia (VP), Coffea Arabica (CA), Rattus norvegicus (RN), Mus musculus (MM), Crenarchaeote (CREN), Mycobacterium vanbaleeni (MV), or Schizosaccharomyces pombe (SP).

FIG. 4 shows the levels of vanillin glucoside, vanillin, vanillyl alcohol glucoside and vanillyl alcohol in yeast strains expressing Penicillium simplicissium (PS) or Rhodococcus jostii (RJ) vanillyl alcohol oxidase (VAO), which were supplemented with 3 mM vanillyl alcohol.

FIG. 5 shows the levels of vanillic acid, vanillin, and vanillin glucoside in yeast strains expressing N. iowensis ACAR or N. crassa ACAR and of E. coli phosphopantetheinyl transferase (PPTase) or S. pombe PPTase, when grown in medium supplemented with 3 mM vanillic acid.

### DETAILED DESCRIPTION OF THE INVENTION

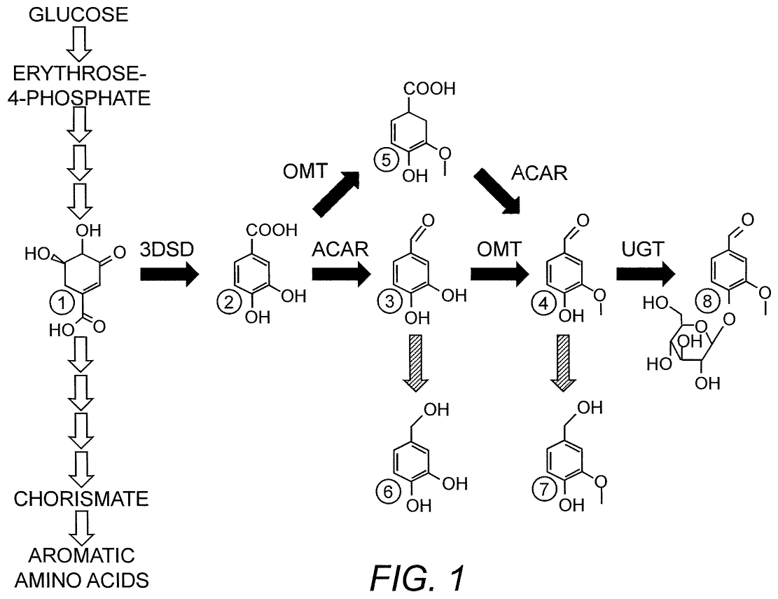

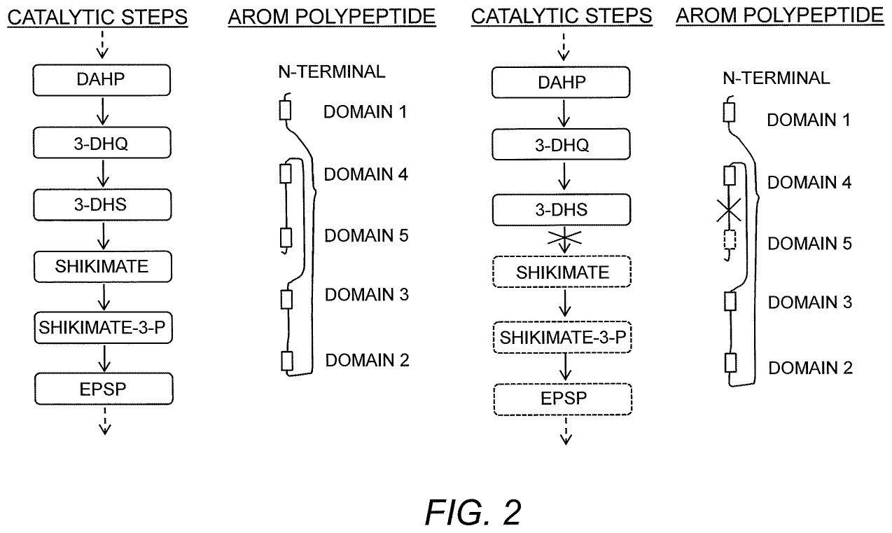

This invention is based on the discovery that mutant AROM polypeptides can be used to increase 3-dehydroshikimate (3-DHS) accumulation in recombinant hosts. 3-DHS is a precursor for vanillin biosynthesis, and if more intracellular 3-DHS is available, more protocatechuic acid can be made in the first committed step of the vanillin biosynthetic pathway. See FIG. 1. AROM is a penta-functional enzyme complex encoded in yeast by the ARO1 gene. The gene is 4764 bp long and encodes a corresponding polypeptide 1588 amino acids in length. AROM performs five consecutive enzymatic conversions, i.e., converting DAHP (3-deoxy-D-arabino-heptulosonic acid-7-phosphate) into 3-DHQ (3-dehydroquinate), which is converted to 3-DHS (3-dehydroshikimic acid), which is converted to shikimate, which is converted to shikimate-3-P (shikimate 3-phosphate), which is converted into EPSP (5-enolpyruvylskimate 3-phosphate), all en route to cellular biosynthesis of the aromatic amino acids tyrosine, tryptophan and phenylalanine (see FIG. 2).

The five catalytic functions of AROM reside on five distinct domains of the ARO1-encoded polypeptide. The functional domains of the AROM polypeptide are in a different order than the order with which they are needed in the five-step conversion of DAHP to EPSP (see FIG. 2). Thus, while domain 1 corresponds with catalytic activity 1, domain 2 corresponds with catalytic activity 5, domain 3 corresponds with catalytic activity 4, domain 4 corresponds with catalytic activity 2, and finally domain 5 corresponds with catalytic activity 3 (see FIG. 1).

Recombinant hosts containing domain 5 variants of the AROM polypeptide (e.g., an AROM polypeptide having one or more mutations within domain 5 or an AROM polypeptide in which a portion of domain 5 is deleted) can have increased levels of 3-DHS in comparison to a corresponding host expressing a wild-type AROM polypeptide. Such hosts also can have increased protocatechuic acid production.

Alternatively, or in addition to, mutant COMT polypeptides can be used to improve biosynthesis of vanillin beta-D-glucoside. For example, mutant COMT polypeptides described herein can have one or more of the following properties: increased turnover; preferential methylation at the meta (3′) position, rather than at the para (4′) position such that production of vanillin is favored over isovanillin; or better specificity for the vanillin pathway substrates, protocatechuic acid and protocatechuic aldehyde.

Accordingly, the invention provides mutant AROM and mutant COMT polypeptides and nucleic acids encoding such polypeptides and use of the same in the biosynthesis of vanillin and/or vanillin glucoside. The method includes the steps of providing a recombinant host capable of producing vanillin in the presence of a carbon source, wherein said recombinant host harbors a heterologous nucleic acid encoding a mutant COMT polypeptide and/or mutant AROM polypeptide; cultivating said recombinant host in the presence of the carbon source; and purifying vanillin and/or vanillin glucoside from said recombinant host or from the cultivation supernatant.

Recombinant hosts described herein can be used in methods to produce vanillin or vanillin glucoside. For example, if the recombinant host is a microorganism, the method can include growing the recombinant microorganism in a culture medium under conditions in which vanillin and/or vanillin glucoside biosynthesis genes are expressed. The recombinant microorganism may be grown in a batch, fed batch or continuous process or combinations thereof. Typically, the recombinant microorganism is grown in a fermentor at a defined temperature(s) in the presence of a suitable nutrient source, e.g., a carbon source, for a desired period of time to produce a desired amount of vanillin and/or vanillin glucoside.

Carbon sources of use in the instant method include any molecule that can be metabolized by the recombinant host cell to facilitate growth and/or production of the vanillin and/or vanillin glucoside. Examples of suitable carbon sources include, but are not limited to, sucrose (e.g., as found in molasses), fructose, xylose, ethanol, glycerol, glucose, cellulose, starch, cellobiose or other glucose containing polymer. In embodiments employing yeast as a host, for example, carbons sources such as sucrose, fructose, xylose, ethanol, glycerol, and glucose are suitable. The carbon source can be provided to the host organism throughout the cultivation period or alternatively, the organism can be grown for a period of time in the presence of another energy source, e.g., protein, and then provided with a source of carbon only during the fed-batch phase.

In one embodiment, the microorganism of this method harbors a nucleic acid encoding mutant AROM polypeptide and optionally a wild-type COMT polypeptide. In another embodiment, the microorganism of this method harbors a nucleic acid encoding mutant COMT polypeptide and optionally a wild-type AROM polypeptide. In yet another embodiment, the microorganism of this method harbors a nucleic acid encoding mutant AROM polypeptide and optionally a mutant COMT polypeptide. Depending on the particular microorganism used in the method, other recombinant genes encoding a 3DSD, ACAR, UGT, or PPTase may also be present and expressed. Thus, the recombinant host may, in one embodiment, be a microorganism harboring one or more heterologous nucleic acids encoding 3DSD, ACAR, UGT or PPTase or a functional homologue of any of the aforementioned sharing at least 80%, such as at least 90%, for example at least 95% sequence identity therewith. Levels of products, substrates and intermediates, e.g., dehydroshikimic acid, protocatechuic acid, protocatechuic aldehyde, vanillin, and vanillin beta-D-glucoside can be determined by extracting samples from culture media for analysis according to published methods.

After the recombinant microorganism has been grown in culture for the desired period of time, vanillin and/or vanillin beta-D-glucoside can then be recovered from the culture using various techniques known in the art, e.g., isolation and purification by extraction, vacuum distillation and multi-stage re-crystallization from aqueous solutions and ultrafiltration (Böddeker, et al. (1997) J. Membrane Sci. 137:155-158; Borges da Silva, et al. (2009) Chem. Eng. Des. 87:1276-1292). Two-phase extraction processes, employing either sulphydryl compounds, such as dithiothreitol, dithioerythritol, glutathione, or L-cysteine (U.S. Pat. No. 5,128,253), or alkaline KOH solutions (WO 94/13614), have been used in the recovery of vanillin as well as for its separation from other aromatic substances. Vanillin adsorption and pervaporation from bioconverted media using polyether-polyamide copolymer membranes has also been described (Boddeker, et al. (1997) supra; Zucchi, et al. (1998) J. Microbiol. Biotechnol. 8:719-722). Macroporous adsorption resins with crosslinked-polystyrene framework have also been used to recover dissolved vanillin from aqueous solutions (Zhang, et al. (2008) Eur. Food Res. Technol. 226:377 383). Ultrafiltration and membrane contactor (MC) techniques have also been evaluated to recover vanillin (Zabkova, et al. (2007) J. Membr. Sci. 301:221-237; Scuibba, et al. (2009) Desalination 241:357-364). Alternatively, conventional techniques such as percolation or supercritical carbon dioxide extraction and reverse osmosis for concentration could be used. If the recombinant host is a plant or plant cells, vanillin or vanillin glucoside can be extracted from the plant tissue using various techniques known in the art.

In some embodiments, vanillin or vanillin beta-D-glucoside can be produced using whole cells that are fed raw materials that contain precursor molecules. The raw materials may be fed during cell growth or after cell growth. The whole cells may be in suspension or immobilized. The whole cells may be in fermentation broth or in a reaction buffer. In some embodiments a permeabilizing agent may be required for efficient transfer of substrate into the cells.

In some embodiments, the vanillin or vanillin beta-D-glucoside is isolated and purified to homogeneity (e.g., at least 90%, 92%, 94%, 96%, or 98% pure). In other embodiments, the vanillin or vanillin beta-D-glucoside is isolated as an extract from a recombinant host. In this respect, vanillin or vanillin beta-D-glucoside may be isolated, but not necessarily purified to homogeneity. Desirably, the amount of vanillin or vanillin beta-D-glucoside produced can be from about 1 mg/l to about 20,000 mg/L or higher. For example about 1 to about 100 mg/L, about 30 to about 100 mg/L, about 50 to about 200 mg/L, about 100 to about 500 mg/L, about 100 to about 1,000 mg/L, about 250 to about 5,000 mg/L, about 1,000 to about 15,000 mg/L, or about 2,000 to about 10,000 mg/L of vanillin or vanillin beta-D-glucoside can be produced. In general, longer culture times will lead to greater amounts of product. Thus, the recombinant microorganism can be cultured for from 1 day to 7 days, from 1 day to 5 days, from 3 days to 5 days, about 3 days, about 4 days, or about 5 days.

It will be appreciated that the various genes and modules discussed herein can be present in two or more recombinant microorganisms rather than a single microorganism. When a plurality of recombinant microorganisms is used, they can be grown in a mixed culture to produce vanillin and/or vanillin glucoside.

Extracts of isolated, and optionally purified, vanillin or vanillin beta-D-glucoside find use in flavoring consumables such as food products, dietary supplements, nutraceuticals, pharmaceutical compositions, dental hygienic compositions, and cosmetic products.

The phrase “food product,” as used herein, includes, but is not limited to, fruits, vegetables, juices, meat products such as ham, bacon and sausage; egg products, fruit concentrates, gelatins and gelatin-like products such as jams, jellies, preserves, and the like; milk products such as ice cream, sour cream and sherbet; icings, syrups including molasses; corn, wheat, rye, soybean, oat, rice and barley products, nut meats and nut products, cakes, cookies, confectionaries such as candies, gums, fruit flavored drops, and chocolates, chewing gum, mints, creams, icing, ice cream, pies and breads, beverages such as coffee, tea, carbonated soft drinks, such as COKE and PEPSI, non-carbonated soft drinks, juices and other fruit drinks, sports drinks such as GATORADE, coffee, teas, iced teas, cola, alcoholic beverages, such as beers, wines and liquors, and KOOL-AID.

Food products also include condiments such as herbs, spices and seasonings, flavor enhancers. A food product also includes prepared packaged products, such as dietetic sweeteners, liquid sweeteners, granulated flavor mixes which upon reconstitution with water provide non-carbonated drinks, instant pudding mixes, instant coffee and tea, coffee whiteners, malted milk mixes, pet foods, livestock feed, tobacco, and materials for baking applications, such as powdered baking mixes for the preparation of breads, cookies, cakes, pancakes, donuts and the like. Food products also include diet or low-calorie food and beverages containing little or no sucrose. Other examples of food products envisioned in accordance with the present invention are described below and throughout the specification.

In another embodiment, the food products are fruits, vegetables, juices, meat products such as ham, bacon and sausage; egg products, fruit concentrates, gelatins and gelatin-like products such as jams, jellies, preserves, and the like; milk products such as ice cream, sour cream and sherbet; icings, syrups including molasses; corn, wheat, rye, soybean, oat, rice and barley products, nut meats and nut products, cakes, cookies, confectionaries such as candies, gums, fruit flavored drops, and chocolates, creams, icing, ice cream, pies and breads.

In another embodiment, the consumable is a pharmaceutical composition. Preferred compositions are pharmaceutical compositions containing vanillin and/or vanillin beta-D-glucoside and one or more pharmaceutically acceptable excipients. These pharmaceutical compositions can be used to formulate pharmaceutical drugs containing one or more active agents that exert a biological effect. As such, the pharmaceutical composition preferably further include one or more active agents that exert a biological effect. Such active agents include pharmaceutical and biological agents that have an activity. Such active agents are well known in the art. See, e.g., The Physician's Desk Reference. Such compositions can be prepared according to procedures known in the art, for example, as described in Remington's Pharmaceutical Sciences, Mack Publishing Co., Easton, PA, USA. In one embodiment, such an active agent includes bronchodilators, anorexiants, antihistamines, nutritional supplements, laxatives, analgesics, anesthetics, antacids, 112-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof. In one embodiment, the active agent is an antipyretics or analgesics, e.g., ibuprofen, acetaminophen, or aspirin; laxatives, e.g., phenolphthalein dioctyl sodium sulfosuccinate; appetite depressants, e.g., amphetamines, phenylpropanolamine, phenylpropanolamine hydrochloride, or caffeine; antacidics, e.g., calcium carbonate; antiasthmatics, e.g., theophylline; antidiuretics, e.g., diphenoxylate hydrochloride; agents active against flatulence, e.g., simethecon; migraine agents, e.g., ergotaminetartrate; psychopharmacological agents, e.g., haloperidol; spasmolytics or sedatives, e.g., phenobarbital; antihyperkinetics, e.g., methyldopa or methylphenidate; tranquilizers, e.g., benzodiazepines, hydroxinmeprobramates or phenothiazines; antihistaminics, e.g., astemizol, chloropheniramine maleate, pyridamine maleate, doxlamine succinate, bromopheniramine maleate, phenyltoloxamine citrate, chlorocyclizine hydrochloride, pheniramine maleate, and phenindamine tartrate; decongestants, e.g., phenylpropanolamine hydrochloride, phenylephrine hydrochloride, pseudoephedrine hydrochloride, pseudoephedrine sulfate, phenylpropanolamine bitartrate, and ephedrine; beta-receptor blockers, e.g., propanolol; agents for alcohol withdrawal, e.g., disulfiram; antitussives, e.g., benzocaine, dextromethorphan, dextromethorphan hydrobromide, noscapine, carbetapentane citrate, and chlophedianol hydrochloride; fluorine supplements, e.g., sodium fluoride; local antibiotics, e.g., tetracycline or cleocine; corticosteroid supplements, e.g., prednisone or prednisolone; agents against goiter formation, e.g., colchicine or allopurinol; antiepileptics, e.g., phenytoine sodium; agents against dehydration, e.g., electrolyte supplements; antiseptics, e.g., cetylpyridinium chloride; NSAIDs, e.g., acetaminophen, ibuprofen, naproxen, or salts thereof; gastrointestinal active agents, e.g., loperamide and famotidine; various alkaloids, e.g., codeine phosphate, codeine sulfate, or morphine; supplements for trace elements, e.g., sodium chloride, zinc chloride, calcium carbonate, magnesium oxide, and other alkali metal salts and alkali earth metal salts; vitamins; ion-exchange resins, e.g., cholestyramine; cholesterol-depressant and lipid-lowering substances; antiarrhythmics, e.g., N-acetylprocainamide; and expectorants, e.g., guaifenesin.

Active substances which have a particularly unpleasant taste include antibacterial agents such as ciprofloxacin, ofloxacin, and pefloxacin; antiepileptics such as zonisamide; macrolide antibiotics such as erythromycin; beta-lactam antibiotics such as penicillins and cephalosporins; psychotropic active substances such as chlorpromazine; active substances such as sulpyrine; and agents active against ulcers, such as cimetidine.

The pharmaceutical compositions of this invention are administered to a subject in any form suitable to achieve their intended purpose. Preferably, however, the composition is one which can be administered buccally or orally. Alternatively, the pharmaceutical composition can be an oral or nasal spray. The subject is any animal, such as a human, although the invention is not intended to be so limited. Other suitable animals include canines, felines, dogs, cats, livestock, horses, cattle, sheep, and the like. A veterinary composition, as used herein, refers to a pharmaceutical composition that suitable for non-human animals. Such veterinary compositions are known in the art.

In another embodiment, the pharmaceutical composition is a liquid dosage form for oral administration, including pharmaceutically acceptable emulsions, solutions, suspensions, syrups, and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1, 3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

The pharmaceutical composition of the present invention can be in the form of a chewable tablet. Chewable tablets are known in the art. See, e.g., U.S. Pat. Nos. 4,684,534 and 6,060,078, each of which is incorporated by reference in its entirety. Any kind of medicament can be contained in the chewable tablet, preferably a medicament of bitter taste, natural plant extracts or other organic compounds. More preferably, vitamins such as vitamin A, vitamin B, vitamin B1, vitamin B2, vitamin B6, vitamin C, vitamin E and vitamin K; natural plant extracts such as Sohgunjung-tang extracts, Sipchundaebo-tang extracts and Eleutherococcus senticosus extracts; organic compounds such as dimenhydrinate, meclazine, acetaminophen, aspirin, phenylpropanolamine, and cetylpyridinium chloride; or gastrointestinal agents such as dried aluminum hydroxide gel, domperidone, soluble azulene, L-glutamine and hydrotalcite can be contained in the core.

The pharmaceutical composition of the present invention can be an orally disintegrating composition. Orally disintegrating tablets are known in the art. See, e.g., U.S. Pat. Nos. 6,368,625 and 6,316,029, each of which is hereby incorporated by reference in its entirety.

The pharmaceutical composition of the present invention can be a solid dosage form, including vanillin or vanillin beta-D-glucoside and a water and/or saliva activated effervescent granule, such as one having a controllable rate of effervescence. The effervescent composition can further comprise a pharmaceutically active compound. Effervescent pharmaceutical compositions are known in the art. See, e.g., U.S. Pat. No. 6,649,186, which is incorporated by reference in its entirety. The effervescent composition can be used in pharmaceutical, veterinary, horticultural, household, food, culinary, pesticidal, agricultural, cosmetic, herbicidal, industrial, cleansing, confectionery and flavoring applications. Formulations incorporating the effervescent composition containing vanillin or vanillin beta-D-glucoside can further include one or more additional adjuvants and/or active ingredients which can be chosen from those known in the art, including flavors, diluents, colors, binders, filler, surfactant, disintegrant, stabilizer, compaction vehicles, and non-effervescent disintegrants.

The pharmaceutical composition can be a film-shaped or wafer-shaped pharmaceutical composition. Such a film-shaped or wafer-shaped pharmaceutical composition can be configured, for example, as quickly disintegrating administration forms, e.g., administration forms disintegrating within a period of 1 second up to 3 minutes, or as slowly disintegrating administration forms, e.g., administration forms disintegrating within a period of 3 to 15 minutes. The indicated disintegration times can be set to the above-mentioned ranges by using, for example, matrix-forming polymers which have different disintegrating, or solubility, characteristics. Thus, by mixing the corresponding polymer components, the disintegration time can be adjusted. In addition, disintegrants are known which “draw” water into the matrix and cause the matrix to burst open from within. As a consequence, certain embodiments of the invention include such disintegrants for the purpose of adjusting the disintegration time.

Suitable are polymers for use in the film-shaped or wafer-shaped pharmaceutical composition include cellulose derivatives, polyvinyl alcohol (e.g. MOWIOL), polyacrylates, polyvinyl pyrrolidone, cellulose ethers, such as ethyl cellulose, as well as polyvinyl alcohol, polyurethane, polymethacrylates, polymethyl methacrylates and derivatives and copolymerizates of the aforementioned polymers.

In certain embodiments, the total thickness of the film-shaped or wafer-shaped pharmaceutical composition according to the invention is preferably 5 μm up to 10 mm, preferably 30 μm to 2 mm, and with particular preference 0.1 mm to 1 mm. The pharmaceutical preparations can be round, oval, elliptic, triangular, quadrangular or polygonal shape, but they can also have any rounded shape.

The pharmaceutical composition of the present invention can be in the form of an aerosol. The aerosol composition can further include a pharmaceutically active agent. Aerosol compositions are known in the art. See, e.g., U.S. Pat. No. 5,011,678, which is hereby incorporated by reference in its entirety. As a nonlimiting example, an aerosol composition according to the present invention can include a medically effective amount of a pharmaceutically active substance, vanillin or vanillin beta-D-glucoside and a biocompatible propellant, such as a (hydro/fluoro) carbon propellant.

In one embodiment of the present invention, the pharmaceutical composition is a nutritional composition. Examples of nutritional compositions having an undesirable taste include, but are not necessarily limited to, enteral nutrition products for treatment of nutritional deficit, trauma, surgery, Crohn's disease, renal disease, hypertension, obesity and the like, to promote athletic performance, muscle enhancement or general well being or inborn errors of metabolism such as phenylketonuria. In particular, such nutritional formulations can contain one or more amino acids which have a bitter or metallic taste or aftertaste. Such amino acids include, but are not limited to, an essential amino acids such as an L isomer of leucine, isoleucine, histidine, lysine, methionine, phenylalanine, threonine, tryptophan, tyrosine or valine.

In one embodiment, the consumable of the present invention is a dental hygienic composition containing vanillin and/or vanillin beta-D-glucoside. Dental hygienic compositions are known in the art and include, but are not necessarily limited to, toothpaste, mouthwash, plaque rinse, dental floss, dental pain relievers (such as ANBESOL), and the like.

In another embodiment, the consumable of the present invention is a cosmetic product containing vanillin and/or vanillin beta-D-glucoside. For example, but not by way of limitation, the cosmetic product can be a face cream, lipstick, lip gloss, and the like. Other suitable compositions of the invention include lip balm, such as CHAPSTICK or BURT'S BEESWAX Lip Balm, further containing vanillin and/or vanillin beta-D-glucoside.

### Arom Multifunctional Enzyme (AROM) Polypeptides

Non-limiting examples of AROM polypeptides include the Saccharomyces cerevisiae polypeptide having the amino acid sequence set forth in SEQ ID NO: 4 (GENBANK Accession No. X06077); a Schizosaccharomyces pombe polypeptide (GENBANK Accession No. NP_594681.1); a Schizosaccharomyces japonicas polypeptide (GENBANK Accession No. XP_002171624); a Neurospora crassa polypeptide (GENBANK Accession No. XP_956000); and a Yarrowia lipolytica polypeptide (GENBANK Accession No. XP_505337).

The term “AROM polypeptide” as used herein refers to any amino acid sequence that is at least 80 percent (e.g., at least 85, 90, 95, 96, 97, 98, 99, or 100 percent) identical to the sequence set forth in SEQ ID NO: 4, and possesses at least four of the five enzymatic activities of the S. cerevisiae AROM polypeptide, i.e., 3-dehydroquinate dehydratase activity, 3-dehydroquinate synthase activity, 3 phosphoshikimate 1-carboxyvinyltransferase activity, shikimate 3-dehydrogenase (NADP+) activity, and shikimate kinase activity.

According to one embodiment of this invention, the AROM polypeptide is a mutant AROM polypeptide with decreased shikimate dehydrogenase activity. When expressed in a recombinant host, the mutant AROM polypeptide redirects metabolic flux from aromatic amino acid production to vanillin precursor production (FIG. 2). Decreased shikimate dehydrogenase activity can be inferred from the accumulation of dehydroshikimic acid in a recombinant host expressing a mutant AROM polypeptide, using LC-MS.

The mutant AROM polypeptide described herein can have one or more modifications in domain 5 (e.g., a substitution of one or more amino acids, a deletion of one or more amino acids, insertions of one or more amino acids, or combinations of substitutions, deletions, and insertions). For example, a mutant AROM polypeptide can have a deletion in at least a portion of domain 5 (e.g., a deletion of the entire domain 5, i.e., amino acids 1305 to 1588 of the amino acid sequence in SEQ ID NO:4, or can have one or more amino acid substitutions in domain 5, such that the mutant AROM polypeptide has decreased shikimate dehydrogenase activity. An exemplary mutant AROM polypeptide lacking domain 5 is provided in SEQ ID NO:2. Exemplary mutant AROM polypeptides with at least one amino acid substitution in domain 5 include the AROM polypeptides A1533P, P1500K, R1458W, V1349G, T1366G, I1387H, W1571V, T1392K, K1370L and A1441P as set forth in SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22 and SEQ ID NO:24, respectively.

Amino acid substitutions that are particularly useful can be found at, for example, one or more positions aligning with position 1349, 1366, 1370, 1387, 1392, 1441, 1458, 1500, 1533, or 1571 of the amino acid sequence set forth in SEQ ID NO:4. For example, a modified AROM polypeptide can have a substitution at a position aligning with position 1370 or at position 1392 of the amino acid sequence set forth in SEQ ID NO:4.

For example, a modified AROM polypeptide can have one or more of the following: an amino acid other than valine (e.g., a glycine) at a position aligning with position 1349 of the amino acid sequence set forth in SEQ ID NO:4; an amino acid other than threonine (e.g., a glycine) at a position aligning with position 1366 of the amino acid sequence set forth in SEQ ID NO:4; an amino acid other than lysine (e.g., leucine) at a position aligning with position 1370 of the amino acid sequence set forth in SEQ ID NO:4; an amino acid other than isoleucine (e.g., histidine) at a position aligning with position 1387 of the amino acid sequence set forth in SEQ ID NO:4; an amino acid other than threonine (e.g., lysine) at a position aligning with position 1392 of the amino acid sequence set forth in SEQ ID NO:4; an amino acid other than alanine (e.g., proline) at a position aligning with position 1441 of the amino acid sequence set forth in SEQ ID NO:4; an amino acid other than arginine (e.g., tryptophan) at a posit aligning with pos 1458 of the amino sequence set forth in ID NO:4; an amino acid other than proline (e.g., lysine) at a position aligning with position 1500 of the amino acid sequence set forth in SEQ ID NO:4; an amino acid other than alanine (e.g., proline) at a position aligning with position 1533 of the amino acid sequence set forth in SEQ ID NO:4; or an amino acid other than tryptophan (e.g., valine) at a position aligning with pos ion 1571 of the amino acid sequence set forth in SEQ ID NO: 4.

In some embodiments, a modified AROM polypeptide is fused to a polypeptide catalyzing the first committed step of vanillin biosynthesis, 3-dehydroshikimate dehydratase (3DSD). A polypeptide having 3DSD activity and that is suitable for use in a fusion polypeptide includes the 3DSD polypeptide from Podospora pauciseta, Ustilago maydis, Rhodoicoccus jostii, Acinetobacter sp., Aspergillus niger or Neurospora crassa. See, GENBANK Accession Nos. CAD60599), XP 001905369.1, XP 761560.1, ABG93191.1, AAC37159.1, and XM 001392464.

For example, a modified AROM polypeptide lacking domain 5 can be fused to a polypeptide having 3DSD activity (e.g., a Podospora pauciseta 3DSD). SEQ ID NO:26 sets forth the amino acid sequence of such a protein and SEQ ID NO:27 sets forth the nucleic acid sequence encoding the protein.

### Catechol-O-Methyl Transferase (COMT) Polypeptides

The COMT polypeptide according to the invention may, in certain embodiments be a caffeoyl-O-methyltransferase. In other embodiments, the COMT polypeptide is preferably a catechol-O-methyltransferase. More preferably, a COMT polypeptide of the invention is a mutant (COMT) polypeptide having improved meta hydroxyl methylation of protocatechuic aldehyde, protocatechuic acid and/or protocatechuic alcohol relative to that of the Homo sapiens COMT having the amino acid sequence set forth in SEQ ID NO:27.

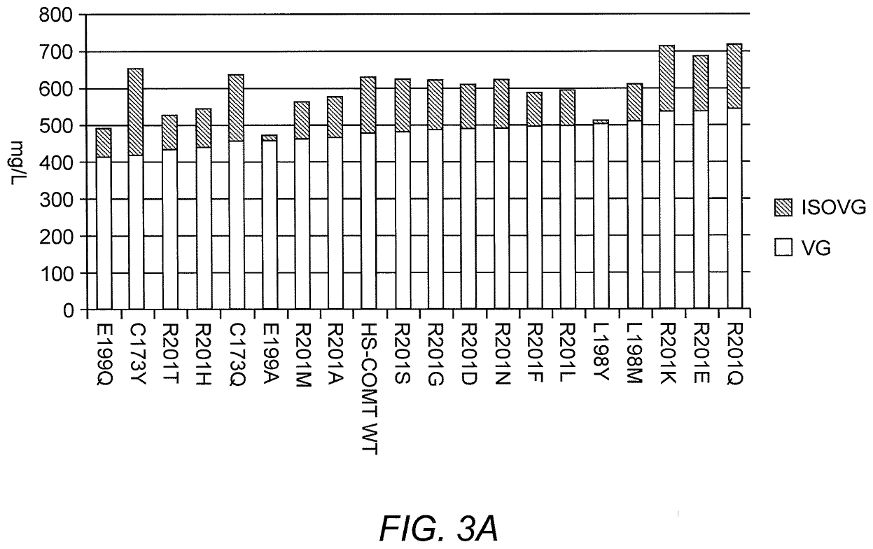

Non-limiting examples of COMT polypeptides that can be mutated in accordance with this invention include COMT polypeptides in the family classified under EC number 2.1.1.6, such as the Homo sapiens (Hs) polypeptide having the amino acid sequence set forth in SEQ ID NO:27 (see, also, GENBANK Accession No. NM_000754); an Arabidopsis thaliana polypeptide having the amino acid sequence set forth in SEQ ID NO:53 (GENBANK Accession No. AY062837); or a Fragaria×ananassa (strawberry) polypeptide having the amino acid sequence set forth in SEQ ID NO:54 (GENBANK Accession No. AF220491). Human COMT polypeptide exists as several variants and the COMT polypeptide may be any of these variants, however in a preferred embodiment the human COMT polypeptide is SEQ ID NO:27 or SEQ ID NO:55. Other suitable mammalian COMT polypeptides of use in this invention include, but are not limited to, those isolated from Pan troglodytes (GENBANK Accession No. XP_514984), Macaca mulatta (GENBANK Accession No. AFJ70145), Equus caballus (GENBANK Accession No. NP_001075303), Canis lupus familiaris (GENBANK Accession No. AAR20324), Cricetulus griseus (GENBANK Accession No. EGV97595), Sus scrofa (GENBANK Accession No. NP_001182259), and Bos taurus (GENBANK Accession No. NP 001095787). Other exemplary COMT polypeptides from plant and microorganism sources include, but are not limited to, those isolated from Rosa chinensis (GENBANK Accession No. CAD29457), Prunus dulcis (GENBANK Accession No. CAA58218), Gossypium hirsutum (GENBANK Accession No. ACT32028), Jatropha curcas (GENBANK Accession No. ACT87981), Eucalyptus camaldulensis (ADB82906), Candida orthopsilosis (GENBANK Accession No. CCG25047), Pichia stipitis (GENBANK Accession No. ABN67921), and Spathaspora passalidarum (GENBANK Accession No. EGW29958). In certain embodiments, the COMT polypeptide of the invention is obtained from Phytophthera infestans (GENBANK Accession No. XP_002899214), Catharanthus roseus (GENBANK Accession No. EGS21863), Yarrowia lipolytica (GENBANK Accession No. XP_500451), Ciona intestinalis (GENBANK Accession No. XP_002121420 or XP_002131313), Capsasproa owczarzaki (GENBANK Accession No. EFW46044), Chaetomium thermophilum (GENBANK Accession No. EGS21863), Clavispora lusitaniae (GENBANK Accession No. XP_002899214), Paracoccidioides sp. ‘lutzii’ Pb01 (GENBANK Accession No. XP_002793380), Vanilla planifolia (SEQ ID NO:56), Coffea Arabica (GENBANK Accession No. AANO3726), Rattus norvegicus (GENBANK Accession No. NP_036663), Mus musculus (GENBANK Accession No. NP_031770), Crenarchaeote (GENBANK Accession No. ABZ07345), Mycobacterium vanbaleeni (GENBANK Accession No. ABM14078), or Schizosaccharomyces pombe (GENBANK Accession No. NP_001018770, which has been shown to exhibit the desired COMT activity (FIG. 3B and FIG. 3C).

The term “COMT polypeptide” as used herein refers to any amino acid sequence that is at least 80 percent (e.g., at least 85, 90, 95, 96, 97, 98, 99, or 100 percent) identical to the Hs COMT sequence set forth in SEQ ID NO:27 and possesses the catechol-O-methyltransferase enzymatic activities of the wild-type Hs COMT polypeptide.

In one embodiment, the term “mutant COMT polypeptide,” as used herein, refers to any polypeptide having an amino acid sequence which is at least 80 percent, such as at least 85 percent, for example at least 90 percent, such as at least 95 percent, for example at least 96 percent, such as at least 97 percent, for example at least 98 percent, such as at least 99 percent identical to the Hs COMT sequence set forth in SEQ ID NO:27 and is capable of catalyzing methylation of the —OH group at the meta position of protocatechuic acid and/or protocatechuic aldehyde, wherein the amino acid sequence of said mutant COMT polypeptide differs from SEQ ID NO:27 by at least one amino acid. In addition, the amino acid sequence of the mutant COMT polypeptide should differ from SEQ ID NO:53, SEQ ID NO:54 and SEQ ID NO:55 by at least one amino acid. It is preferred that the mutant COMT polypeptide differs by at least one amino acid from any sequence of any wild type COMT polypeptide.

In another embodiment of the invention, the term “mutant COMT polypeptide” refers to a polypeptide having an amino acid sequence, which is at least 80 percent, such as at least 85 percent, for example at least 90 percent, such as at least 95 percent, for example at least 96 percent, such as at least 97 percent, for example at least 98 percent, such as at least 99 percent identical to either SEQ ID NO:53 or SEQ ID NO:54 and is capable of catalyzing methylation of the —OH group at the meta position of protocatechuic acid and/or protocatechuic aldehyde, wherein the amino acid sequence of said mutant COMT polypeptide differs from each of SEQ ID NO:53 and SEQ ID NO:54 by at least one amino acid.

The mutant COMT polypeptides described herein can have one or more mutations (e.g., a substitution of one or more amino acids, a deletion of one or more amino acids, insertions of one or more amino acids, or combinations of substitutions, deletions, and insertions) in, for example, the substrate binding site. For example, a mutant COMT polypeptide can have one or more amino acid substitutions in the substrate binding site of human COMT.

In certain embodiments, a “mutant COMT polypeptide” of the invention differs from SEQ ID N0:27, SEQ ID N0:53, SEQ ID N0:54 or SEQ ID N0:55 by only one or two amino acid residues, wherein the differences between said mutant and wild-type proteins are in the substrate bind site.

As described herein, mutant COMT polypeptides can be used to improve biosynthesis of vanillin glucoside. For example, mutant COMT polypeptides can have one or more of the following properties: increased turnover; preferential methylation at the meta (3′) position, rather than at the para (4′) position such that production of vanillin is favored over isovanillin; or better specificity for the vanillin pathway substrates, protocatechuic acid and protocatechuic aldehyde. Mutant COMT polypeptides can be characterized in vitro using methylation assays or characterized in vivo in a recombinant host based on production of vanillic acid, vanillin, or vanillin glucoside.

The structures of iso-vanillin, vanillin, vanillic acid and vanillic acid are as follows.

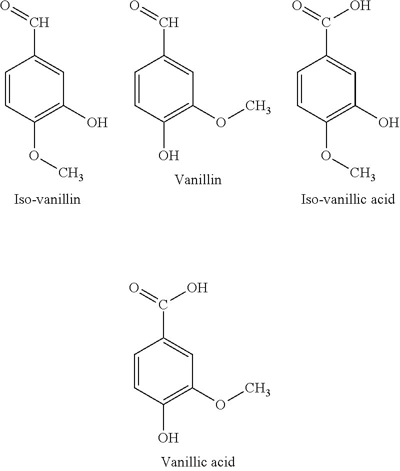

The wild-type Hs COMT lacks regioselective O-methylation of protocatechuic aldehyde and protocatechuic acid, indicating that the binding site of Hs COMT does not bind these substrates in an orientation that allows the desired regioselective methylation. Without being bound to a particular mechanism, the active site of Hs COMT is composed of the co-enzyme S-adenosyl methionine (SAM), which serves as the methyl donor, and the catechol substrate, which contains the hydroxyl to be methylated coordinated to Mg²⁺ and proximal to Lys144. The O-methylation proceeds via an SN2 mechanism, where Lys144 serves as a catalytic base that deprotonates the proximal hydroxyl to form the oxy-anion that attacks a methyl group from the sulfonium of SAM. See, for example, Zheng & Bruice (1997) J Am. Chem. Soc. 119(35):8137-8145; Kuhn & Kollman (2000) J. Am. Chem. Soc. 122(11):2586-2596; Roca, et al. (2003) J. Am. Chem. Soc. 125(25):7726-37.

In one embodiment of the invention the invention provides a mutant COMT polypeptide, which is capable of catalyzing methylation of an —OH group of protocatechuic acid, wherein said methylation results in generation of at least 4 times more vanillic acid compared to iso-vanillic acid, preferably at least 5 times more vanillic acid compared to iso-vanillic acid, such as at least 10 times more vanillic acid compared to iso-vanillic acid, for example at least 15 times more vanillic acid compared to iso-vanillic acid, such as at least 20 times more vanillic acid compared to iso-vanillic acid, for example at least 25 times more vanillic acid compared to iso-vanillic acid, such as at least 30 times more vanillic acid compared to iso-vanillic acid; and which has an amino sequence which differs from SEQ ID NO:27 by at least one amino acid.

In addition to furthermore preferred above mentioned properties, it is that a mutant COMT polypeptide is capable of catalyzing methylation of an —OH group of protocatechuic aldehyde, wherein said methylation results in generation of at least 4, 5, 10, 15, 20, 25, or 30 times more vanillin compared to iso-vanillin; and/or is capable of catalyzing methylation of an —OH group of protocatechuic alcohol, wherein said methylation results in generation of at least 4, 5, 10, 15, 20, 25, or 30 times more vanillyl alcohol compared to iso-vanillyl alcohol.

To determine whether a given mutant COMT polypeptide is capable of catalyzing methylation of an —OH group of protocatechuic acid, wherein said methylation results in generation of at least X times more vanillic acid compared to iso-vanillic acid, an in vitro assay can be conducted. In such an assay, protocatechuic acid is incubated with a mutant COMT polypeptide in the presence of a methyl donor and subsequently the level of generated iso-vanillic acid and vanillic acid is determined. Said methyl donor may for example be S-adenosylmethionine. More preferably, this may be determined by generating a recombinant host harboring a heterologous nucleic acid encoding the mutant COMT polypeptide to be tested, wherein said recombinant host furthermore is capable of producing protocatechuic acid. After cultivation of the recombinant host, the level of generated iso-vanillic acid and vanillic acid may be determined. In relation to this method it is preferred that said heterologous nucleic acid encoding the mutant COMT polypeptide to be tested is operably linked to a regulatory region allowing expression in said recombinant host. Furthermore, it is preferred that the recombinant host expresses at least one 30S0 and at least one ACAR, which preferably may be one of the 3DSD's and ACAR's described herein. In embodiments where the recombinant host expresses an ACAR capable of catalyzing conversion of vanillic acid to vanillin, then the method may also include determining the level of generated vanillin and iso-vanillin. The recombinant host may also express at least one UGT capable of catalyzing glucosylation of vanillin and isovanillin, in which case the levels of vanillin-glucoside and iso-vanillin-glucoside may be determined instead of the levels of vanillin and iso-vanillin, respectively. Alternatively, this may be determined by generating a recombinant host harboring a heterologous nucleic acid encoding the mutant COMT polypeptide to be tested, and feeding protocatechuic acid to said recombinant host, followed by determining the level of generated iso-vanillic acid and vanillic acid.

Similarly, an in vitro assay or a recombinant host cell can be used to determine whether a mutant COMT polypeptide is capable of catalyzing methylation of an —OH group of protocatechuic aldehyde, wherein said methylation results in generation of at least X times more vanillin compared to iso-vanillin. However, in this assay, protecatechuic aldehyde is used as starting material and the level of vanillin and iso-vanillin is determined.

Likewise, an in vitro assay or a recombinant host cell can be used to determine whether a given mutant COMT polypeptide is capable of catalyzing methylation of an —OH group of protocatechuic alcohol, wherein said methylation results in generation of at least X times more vanillyl alcohol compared to iso-vanillyl alcohol. However, in this assay, protecatechuic alcohol is used as starting material and the level of vanillyl alcohol and iso-vanillyl alcohol is determined.

The level of isovanillin and vanillin may be determined by any suitable method useful for detecting these compounds, wherein said method can distinguish between isovanillin and vanillin. Such methods include for example HPLC. Similarly, the level of iso-vanillic acid, vanillic acid, iso-vanillyl alcohol and vanillyl alcohol may be determined using any suitable method useful for detecting these compounds, wherein said method can distinguish between isovanillin and vanillin. Such methods include for example HPLC.

For substrates of the dimensions of protocatechuic aldehyde and protocatechuic acid, the boundary of the substrate binding site of Hs COMT was found to be formed by the following hydrophobic residues: Trp38, Met40, Cys173, Pro174, Trp143, and Leu198. An additional hydrophilic residue that may influence binding is Arg201. The hydroxyl of the catechol that is not methylated hydrogen bonds with Glu199.

In accordance with this mechanism, for meta-position methylation of protocatechuic aldehyde and protocatechuic acid to occur, these substrates must be bound in an orientation that places the meta hydroxyl so that it is coordinated to Mg²⁺ and proximal to Lys144, whereas the para hydroxyl is proximal to Glu199. The observed lack of this desired regioselectivity in wild-type Hs COMT suggests that this binding orientation does not preferentially occur. One or more amino acids can be substituted for the binding site residues of Hs COMT to allow a binding orientation of the substrates that promotes the desired meta O-methylation of protocatechuic aldehyde and protocatechuic acid.

Therefore, this invention also includes a method for identifying COMT polypeptides with improved substrate specificity. In particular, such a method provides computational methodology for identifying residue mutations that confer improved meta O-methylation by a COMT polypeptide. The method includes several distinct steps. In particular, one method of identifying optimum mutations includes the steps of (a) selecting a protein structure of a COMT polypeptide; (b) docking substrates protocatechuic acid and protocatechuic aldehyde to the protein structure of the COMT polypeptide determined in (a) to deduce the different conformations which promote regioselective meta or para O-methylation; (c) identifying binding site mutations proximal to protocatechuic acid and protocatechuic aldehyde substrates; (d) performing in silica mutational analysis on each residue identified in (c); (e) ranking candidate residue mutations for each position from (d) based on predicted conformations of substrate of meta or para O-methylation; and (f) selecting best scoring mutations for each candidate residue identified in (c).

As discussed above, the COMT polypeptide can be in the COMT family classified under EC number 2.1.1.6, catechol O-methyl transferase. As such, one skilled in the art would appreciate that, in addition to Hs COMT, the method can be applied to any species of COMT within this classification, wherein such proteins will have similar binding site residues. In some embodiments, the method is applied to the Arabidopsis or strawberry COMT (SEQ ID NO:53 and SEQ ID NO:54, respectively). While each of the method steps are described in more detail with respect to Hs COMT, it will be appreciated that similar method steps can be performed with other COMT polypeptides.

In step (a), a COMT protein structure is selected. Protein structures of Hs COMT are publically available from the Protein Data Bank and can be assessed for utility based upon resolution, inclusion of mutations, and other sequence variations that may been introduced to afford crystallization. Other factors that can be considered in choosing a structure include whether or not the structure includes a substrate bound to the protein, or the nature of the substrate bound to the protein. Crystal structure code 3BWM (RCSB Protein Data Bank) is a particularly useful structure for Hs COMT, and was used in the methods described herein. One skilled in the art would appreciate that other Hs COMT crystal structure can be used as inputs to modeling procedures.

In step (b), the substrates of interest, e.g., protocatechuic acid and protocatechuic aldehyde, are docked to the protein structure. Docking is a term denoting a computational approach implemented in a variety of algorithms of predicting a likely conformation of a selected small molecule in a protein binding site. The technique typically calculates a binding score to provide the basis to evaluate the goodness of fit and predicted binding energy of interaction. The docking program used to derive the substrate conformations used in the current procedure is ProtoScreen (Haydon, et al. (2008) Science 321:1673-1675). The method creates binding scores according to the ProtoScore algorithm (Bhurruth-Alcor, et al. (2011) Org. Biomol. Chem. 9:1169-1188). One skilled in the art will appreciate that other docking programs may be used in this method step to identify suitable binding conformations of protocatechuic acid and protocatechuic aldehyde substrates in Hs COMT (or other COMT family member).

In step (c), mutational analysis is performed. This can include several substeps such as (i) identifying a first residue to be mutated; (ii) identifying a residue to mutate to from list of suitable amino acids; (iii) for each residue in (ii), searching a rotamer library for conformational candidates; (iv) mutating the residue from (i) with each of the new residue side chain rotamer selections from (iii); (v) minimizing protein complex conformation; (vi) scoring each rotamer candidate in (iv) with various calculations of mutant-substrate, mutant-protein, mutant-solvent and substrate solvent energies with different conformations of protocatechuic acid or aldehyde allowing for energy comparisons between where the substrate is being modified either at the meta or the para position; and (vii) ranking rotamer selections from (iii) and then highest scoring amino acid mutants from (ii).

In substep (i), each of the residues to be mutated are analyzed in turn.

In substep (ii), a residue to mutate is chosen from a deterministic list of available amino acids. By default this list is the standard 20 amino acids found in nature, but could include other non-natural amino acids.

In substep (iii), all rotamers are identified in a rotamer library that matches the mutant amino acid identity. A rotamer library is a precomputed set of preferred conformations of standard amino acid side chains in a useable 3D format. Such libraries are commonly used in protein structural analysis work, and are included in most commercially available molecular modeling packages. The set of rotamers that match the identity of the mutating residue are selected for substep (iv).

In substep (iv), the selected protein residue is swapped for each of the rotamers identified in (iii). This involves manipulating the computational representation of the protein residue atoms such that the starting residue side chain atoms are deleted before the incoming selected rotamer from (iii) are connected to the alpha carbon position using vector mathematics. This method is repeated for each candidate rotamer to arrive at a list of 3D representations of the protein each varying only by differing rotamer conformations at a single residue position.

In substep (v), the protein-substrate complexes derived in (iv) are subject to forcefield minimizations. In a particularly useful embodiment, this forcefield is AMBER99 with AM1-BCC charges applied to the substrate. In another aspect of the method, a Born solvation term is used. The protein backbone can be tethered using wall constraints, while side chains remain unconstrained. This has the effect of reducing overall protein motion but allowing the effect of the individual residue mutation on neighboring residues to be explored. One skilled in the art will appreciate that various commercially available molecular modeling packages are capable of performing these tasks.

In substep (vi), the resulting protein-complex conformations are subject to energetic calculations to determine the viability of the individual mutant conformations. This includes individually calculating the wild-type residue interaction energy with the substrate, protein environment and solvent and then performing the same calculations with the mutated residue conformation. The calculations are determined for both (meta and para reacting) conformations of the substrate. This method step thus identifies mutations which have favorable binding energies for the substrate in the meta reacting pose compared to the para activating pose. The energies are calculated using forcefield based terms. In one embodiment, the forcefield is AMBER99 and the interaction energy between an amino acid and the protein-substrate environment is described by equation 1.

$${E}_{non - bonded} = {E}_{van - der - Waals} + {E}_{electrostatic} \qquad ( Equation 1 )$$

$$where$$

$${E}_{van - der - Waals} = {\sum }_{nonbonded pairs} ( \frac{{A}_{ik}}{{r}_{ik}^{12}} - \frac{{C}_{ik}}{{r}_{ik}^{6}} )$$

$${E}_{electrostatic} = {\sum }_{nonbonded pairs} \frac{{q}_{\text{?}} {q}_{k}}{D \text{?}}$$

$$\text{?} \text{indicates text missing or illegible when filed}$$

One skilled in the art will appreciate that other similar equations can be implemented to derive interaction energies suitable for this differential energetic analysis of different substrate-mutated amino acid poses.

In substep (vii), mutant amino acids are selected based upon favorable binding energies of substrate bound in the meta reacting predicted pose compared to the para reacting predicted pose. These calculations thus determines which amino acid mutations are likely to promote meta regioselective 0-methylation.

In step (iv), step (iii) is repeated until a list of energy values is outputted for each of the binding site residues to be mutated.

In step (v), the list of energy values derived from step (iv) is ranked by the differential in energy between each mutation with where the substrate is in the meta reacting position compared to the para reacting position. The entries at the top of this list represent where mutations favor meta O-methylation over para O-methylation.

In step (vi), a limited number of such candidates from step (v) are selected based on the energy values. Mutations are not selected where (1) mutations are energetically unfavorable or (2) where mutations are not predicted to alter regioselectivity.

As described herein, application of the method described above to Hs COMT resulted in the identification of a set of mutations that are designed to improve enzyme regioselectivity O-methylation. The mutations can be used independently or in combination.

In the membrane bound isoform of Hs COMT, the equivalent residue numbers are those of soluble Hs COMT plus fifty. Therefore residues described or replaced in soluble Hs COMT are also inferred to be described or replaced at the residue number plus fifty in membrane bound Hs COMT.

In one embodiment, the invention provides a mutant COMT polypeptide, which (1) has an amino acid sequence sharing at least percent, such as at least 85 percent, for example at least 90 percent, such as at least 95 percent, for example at least 96 percent, such as at least 97 percent, for example at least 98 percent, such as at least 99 percent sequence identity with SEQ ID NO:27 determined over the entire length of SEQ ID NO:27; and (2) has at least one amino acid substitution at a position aligning with positions 198 to 199 of SEQ ID NO:27, which may be any of the amino acid substitutions described herein below; and (3) is capable of catalyzing methylation of an —OH group of protocatechuic acid, wherein said methylation results in generation of at least 4, 5, 10, 15, 20, 25 or 30 times more vanillic acid compared to iso-vanillic acid. In addition these characteristics, said mutant COMT polypeptide may also be capable of catalyzing methylation of an —OH group of protocatechuic aldehyde, wherein said methylation results in generation of at least 4, 5, 10, 15, 20, 25 or 30 times more vanillin compared to iso-vanillin; and/or be capable of catalyzing methylation of an —OH group of protocatechuic alcohol, wherein said methylation results in generation of at least 4, 5, 10, 15, 20, 25, or 30 times more vanillyl alcohol compared to iso-vanillyl alcohol.

[Thus, the mutant COMT polypeptide may in one preferred embodiment have an amino acid substitution at the position aligning with position 198 of SEQ ID NO:27. Accordingly, the mutant COMT polypeptide may be a mutant COMT polypeptide with the characteristics outlined above, wherein said substitution is a substitution of the leucine at the position aligning with position 198 of SEQ ID N0:27 with another amino acid having a lower hydropathy index. For example, the mutant COMT polypeptide may be a mutant COMT polypeptide with characteristics as outlined above, wherein said substitution is a substitution of the leucine at the position aligning with position 198 of SEQ ID N0:27 with another amino acid having a hydropathy index lower than 2. Thus, the mutant COMT polypeptide may be a mutant COMT polypeptide with characteristics as outlined above, wherein said substitution is a substitution of the leucine at the position aligning with position 198 of SEQ ID N0:27 with an Ala, Arg, Asn, Asp, Cys, Glu, Gln, Gly, His, Lys, Met, Phe, Pro, Ser, Thr, Trp or Tyr, for example Ala, Arg, Asn, Asp, Glu, Gln, Gly, His, Lys, Met, Pro, Ser, Thr, Trp or Tyr. However, preferably said substitution is a substitution of the leucine at the position aligning with position 198 of SEQ ID N0:27 with tyrosine. Substitution of the leucine aligning with position 198 of SEQ ID N0:27 with methionine increased regioselectivity of meta>para 0-methylation for protocatechuic aldehyde.

In another preferred embodiment, the mutant COMT polypeptide may have an amino acid substitution at the position aligning with position 199 of SEQ ID N0:27. Accordingly, the mutant COMT polypeptide may be a mutant COMT polypeptide with characteristics as outlined above, wherein said substitution is a substitution of the glutamic acid at the position aligning with position 199 of SEQ ID N0:27 with another amino acid, which has either a neutral or positive side-chain charge at pH 7.4. Thus, the mutant COMT polypeptide may be a mutant COMT polypeptide with characteristics as outlined above, wherein said substitution is a substitution of the glutamic acid at the position aligning with position 199 of SEQ ID NO:27 with Ala, Arg, Asn, Cys, Gln, Gly, His, Ile, Leu, Lys, Met, Phe, Pro, Ser, Thr, Trp, Tyr or Val. However, preferably said substitution is a substitution of the glutamic acid at the position aligning with position 199 of SEQ ID NO:27 with an alanine or glutamine. Substitution of the glutamic acid aligning with position 199 of SEQ ID NO:27 with alanine or glutamine increased regioselectivity of meta>para O-methylation for protocatechuic aldehyde.

For example, a mutant COMT polypeptide can have one or more of the following mutations: a substitution of a tryptophan, tyrosine, phenylalanine, glutamic acid, or arginine for the leucine at a position aligning with position 198 of the amino acid sequence set forth in SEQ ID NO:27; a substitution of an arginine, lysine, or alanine for methionine at a position aligning with position 40 of the amino acid sequence set forth in SEQ ID NO:27; a substitution of a tyrosine, lysine, histidine, or arginine for the tryptophan at a position aligning with position 143 of the amino acid sequence set forth in SEQ ID NO:27; a substitution of an isoleucine, arginine, or tyrosine for the proline at a position aligning with position 174 of the amino acid sequence set forth in SEQ ID NO:27; a substitution of an arginine or lysine for tryptophan at a position aligning with position 38 of the amino acid sequence set forth in SEQ ID NO:27; a substitution of a phenylalanine, tyrosine, glutamic acid, tryptophan, or methionine for cysteine at a position aligning with position 173 of the amino acid sequence set forth in SEQ ID NO:27; and/or a substitution of a serine, glutamic acid, or aspartic acid for arginine at a position aligning with position 201 of the amino acid sequence set forth in SEQ ID NO:27.

In one embodiment, a mutant COMT polypeptide contains substitution of tryptophan for leucine at a position aligning with position 198. This mutation may increase regioselectivity of meta>para 0-methylation for protocatechuic acid. Modeling of the protein binding site of a COMT polypeptide containing a L198W mutation, indicates that a steric clash can occur between the mutated residue and the substrate. This steric clash does not occur in the meta reacting conformation as the carboxylic acid of the substrate is distal to this residue.

In another embodiment of the invention, the mutant COMT polypeptide is a polypeptide of SEQ ID N0:27, wherein the amino acid at position 198 has been substituted with an amino acid having a lower hydropathy index than leucine. For example, the mutant COMT polypeptide may be a polypeptide of SEQ ID N0:27, wherein the leucine at the position 198 has been substituted with an amino acid having a hydropathy index lower than 2. Thus, the mutant COMT polypeptide may be a polypeptide of SEQ ID NO:1, wherein the leucine at position 198 has been substituted with an Ala, Arg, Asn, Asp, Glu, Gln, Gly, His, Lys, Met, Pro, Ser, Thr, Trp or Tyr, preferably Met or Tyr.

In another preferred embodiment, the mutant COMT polypeptide may be a polypeptide of SEQ ID N0:27, wherein the amino acid at position 199 has been substituted with another amino acid, which has either a neutral or positive side-chain charge at pH 7.4. Thus, the mutant COMT polypeptide may be a polypeptide of SEQ ID N0:27 where the glutamic acid at the position 199 has been substituted with Ala, Arg, Asn, Cys, Gln, Gly, His, Ile, Leu, Lys, Met, Phe, Pro, Ser, Thr, Trp, Tyr or Val, preferably Ala or Gln.

In some embodiments, a mutant COMT polypeptide has two or more mutations. For example, 2, 3, 4, 5, 6, or 7 of the residues in the substrate binding site can be mutated. For example, in one embodiment, a mutant COMT polypeptide can have a substitution of an arginine or lysine for methionine at a position aligning with position 40 of the amino acid sequence of SEQ ID NO:27; a substitution of a tyrosine or histidine for tryptophan at a position aligning with position 143 of the amino acid sequence of SEQ ID NO:27; a substitution of an isoleucine for proline at a position aligning with position 174 of the amino acid sequence of SEQ ID NO:27, and a substitution of an arginine or lysine for tryptophan at position 38. A mutant COMT polypeptide also can have a substitution of lysine or arginine for tryptophan at a position aligning with position 143 of the amino acid sequence of SEQ ID NO:27 and a substitution of an arginine or tyrosine for proline at position 174 of SEQ ID NO:27. A mutant COMT polypeptide also can have a substitution of a phenylalanine, tyrosine, glutamic acid, tryptophan, or methionine for cysteine at a position aligning with position 173 of the amino acid sequence set forth in SEQ ID NO:27, a substitution of an alanine for methionine at a position aligning with position 40 of the amino acid sequence set forth in SEQ ID NO:27, and a substitution of a serine, glutamic acid, or aspartic acid for the arginine at a position aligning with position 201 of the amino acid sequence set forth in SEQ ID NO:27. It is also possible that the mutant COMT polypeptide has a substitution of the leucine at a position aligning with position 198 of SEQ ID NO:27 as well as a substitution of the glutamic acid at a position aligning with position 199 of SEQ ID NO:27. Said substitutions may be any of the substitutions described in this section above, It is also possible that the mutant COMT polypeptide has a substitution of the leucine at a position aligning with position 198 of SEQ ID NO:27 as well as a substitution of the arginine at a position aligning with position 201 of SEQ ID NO:27. Said substitutions may be any of the substitutions described in this section above.

### Percent Identity

The sequence identities given herein are preferably sequence identity over the entire length of the reference sequence. Accordingly, sequence identity to the amino acid sequence provided as SEQ ID N0:4 or SEQ ID N0:27 herein is sequence identity over the entire length of SEQ ID N0:4 or SEQ ID N0:27, respectively.

Percent identity can be determined as follows. A reference sequence (e.g., a nucleic acid sequence or an amino acid sequence set forth in SEQ ID N0:4 or SEQ ID N0:27) is aligned to one or more candidate sequences using the computer program ClustalW (version 1.83, default parameters), which allows alignments of nucleic acid or polypeptide sequences to be carried out across their entire length (global alignment). Chenna et al. 31(13):3497-500. (2003) Nucleic Acids Res.

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pair-wise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pair-wise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences.

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

### Amino Acid Substitutions

Amino acid substitutions can be conservative or non-conservative. Conservative amino acid substitutions replace an amino acid with an amino acid of the same class, whereas non-conservative amino acid substitutions replace an amino acid with an amino acid of a different class. Examples of conservative substitutions include amino acid substitutions within the following groups: (1) glycine and alanine; (2) valine, isoleucine, and leucine; (3) aspartic acid and glutamic acid; (4) asparagine, glutamine, serine, and threonine; (5) lysine, histidine, and arginine; and (6) phenylalanine and tyrosine.

Non-conservative amino acid substitutions may replace an amino acid of one class with an amino acid of a different class. Non-conservative substitutions can make a substantial change in the charge or hydrophobicity of the gene product. Non-conservative amino acid substitutions also can make a substantial change in the bulk of the residue side chain, e.g., substituting an alanine residue for an isoleucine residue. Examples of non-conservative substitutions include the substitution of a basic amino acid for a non-polar amino acid or a polar amino acid for an acidic amino acid. One of ordinary skill in the art will appreciate that similar amino acids can be substituted for the mutants described herein.

### Nucleic Acids

This document also provides isolated nucleic acids encoding the mutant AROM and COMT polypeptides. An “isolated nucleic acid” refers to a nucleic acid that is separated from other nucleic acid molecules that are present in a genome, including nucleic acids that normally flank one or both sides of the nucleic acid in a genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., any paramyxovirus, retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

Nucleic acids encoding AROM or COMT polypeptides can be modified using common molecular cloning techniques (e.g., site-directed mutagenesis) to generate mutations at particular positions of the encoded polypeptide (e.g., positions aligning with position 1349, 1366, 1370, 1387, 1392, 1441, 1458, 1500, 1533, or 1571 of the AROM amino acid sequence set forth in SEQ ID NO: 4; or positions aligning with position 38, 40, 143, 173, 174, 198, or 201 of the soluble form of the human COMT amino acid sequence set forth in SEQ ID NO:27). Nucleic acid molecules can include a single nucleotide mutation or more than one mutation, or more than one type of mutation. Polymerase chain reaction (PCR) and nucleic acid hybridization techniques can be used to identify nucleic acids encoding AROM polypeptides having altered amino acid sequences.

In some embodiments, a nucleic acid molecule encoding a mutant polypeptide of this invention can include a tag sequence that encodes a “tag” designed to facilitate subsequent manipulation (e.g., to facilitate purification or detection), secretion, or localization of the encoded polypeptide. Tag sequences can be inserted in the nucleic acid sequence encoding the AROM or COMT polypeptide such that the encoded tag is located at either the carboxyl or amino terminus of the AROM or COMT polypeptide. Non-limiting examples of encoded tags include green fluorescent protein (GFP), glutathione S transferase (GST), HIS tag, and FLAG tag (Kodak, New Haven, CT). Other examples of tags include a chloroplast transit peptide, a mitochondrial transit peptide, an amyloplast peptide, signal peptide, or a secretion tag.

Polypeptides (mutant or wild-type) of this invention can be produced using any method. For example, polypeptides can be produced by chemical synthesis. Alternatively, polypeptides described herein can be produced by standard recombinant technology using heterologous expression vectors encoding polypeptides. Expression vectors can be introduced into host cells (e.g., by transformation or transfection) for expression of the encoded polypeptide, which then can be purified. Expression systems that can be used for small or large scale production of polypeptides include, without limitation, microorganisms such as bacteria (e.g., E. coli and B. subtilis) transformed with recombinant bacteriophage DNA, plasmid DNA, or cosmid DNA expression vectors containing the nucleic acid molecules described herein, and yeast (e.g., S. cerevisiae or S. pombe) transformed with recombinant yeast expression vectors containing the nucleic acid molecules described herein. Useful expression systems also include insect cell systems infected with recombinant virus expression vectors (e.g., baculovirus) containing the nucleic acid molecules described herein, and plant cell systems infected with recombinant virus expression vectors (e.g., tobacco mosaic virus) or transformed with recombinant plasmid expression vectors (e.g., Ti plasmid) containing the nucleic acid molecules described herein. Polypeptides of this invention also can be produced using mammalian expression system harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids described herein. Polypeptides of this invention can have an N-terminal or C-terminal tag as discussed above.

### Recombinant Hosts

This invention also features recombinant hosts. As used herein, the term recombinant host is intended to refer to a host, the genome of which has been augmented by at least one incorporated DNA sequence. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that typically the genome of a recombinant host described herein is augmented through the stable introduction of one or more recombinant genes. However, autonomous or replicative plasmids or vectors can also be used within the scope of this invention. Moreover, the present invention can be practiced using a low copy number, e.g., a single copy, or high copy number (as exemplified herein) plasmid or vector.

Generally, the introduced DNA is not originally resident in the host that is the recipient of the DNA, but it is within the scope of the invention to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms, plant cells, and plants.

The term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene may be a DNA sequence from another species, or may be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA.

A recombinant gene encoding a polypeptide described herein includes the coding sequence for that polypeptide, operably linked, in sense orientation, to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. The term “heterologous nucleic acid” as used herein, refers to a nucleic acid introduced into a recombinant host, wherein said nucleic acid is not naturally present in said host. Thus, if the recombinant host is a microorganism, the coding sequence can be from other prokaryotic or eukaryotic microorganisms, from plants or from animals. n some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically includes at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., intrans, enhancers, upstream activation regions, transcription terminators, and inducible elements.

One or more genes, for example one or more heterologous nucleic acids, can be combined in a recombinant nucleic acid construct in “modules” useful for a discrete aspect of vanillin and/or vanillin glucoside production. Combining a plurality of genes or heterologous nucleic acids in a module, facilitates the use of the module in a variety of species. For example, a vanillin gene cluster can be combined such that each coding sequence is operably linked to a separate regulatory region, to form a vanillin module for production in eukaryotic organisms. Alternatively, the module can express a polycistronic message for production of vanillin and/or vanillin glucoside in prokaryotic hosts such as species of Rodobacter, E. coli, Bacillus or Lactobacillus. In addition to genes useful for vanillin or vanillin glucoside production, a recombinant construct typically also contains an origin of replication, and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression appropriate in a codon particular host bias tables for is obtained, that host using (e.g., microorganism). As isolated nucleic acids, these modified sequences can exist as purified molecules and can be incorporated into a vector or a virus for use in constructing modules for recombinant nucleic acid constructs.

Recombinant hosts described herein express mutant AROM polypeptides and/or mutant COMT polypeptides. Thus, in one aspect, the present invention relates to a recombinant host harboring a heterologous nucleic acid encoding a mutant AROM polypeptide and/or a mutant COMT polypeptide, which may be any of the mutant polypeptides described herein. In particular, the invention relates to a recombinant host harboring a heterologous nucleic acid encoding said mutant AROM polypeptide and/or mutant COMT polypeptide, wherein said nucleic acid is operably linked to a regulatory region allowing expression in said recombinant host.

Such hosts further can include additional genes or biosynthetic modules to produce vanillin or vanillin glucoside, improve efficiency with which energy and carbon sources are converted to vanillin and its glucoside, and/or to enhance productivity from the cell culture or plant. Such additional biosynthetic modules can include one or more of a gene encoding a 3DSD polypeptide, a gene encoding a phosphopantetheinyl transferase (PPTase), and a gene encoding a UGT polypeptide. See FIG. 1. These genes may be endogenous genes or recombinant genes. In addition, when the host cell harbors a heterologous nucleic acid encoding a mutant AROM polypeptide, the host cell may further include a wild-type OMT gene. Likewise, when the host cell harbors a heterologous nucleic acid encoding a mutant COMT polypeptide, the host cell may further include a wild-type AROM gene. Alternatively, the host cell may harbor heterologous nucleic acids encoding a mutant COMT polypeptide and a mutant AROM polypeptide, as described herein. In addition, the host can further express a Vanillyl Alcohol Oxidase (VAO) enzyme.

Suitable 3DSD polypeptides are known. A 3DSD polypeptide according to the present invention may be any enzyme with 3-dehydroshikimate dehydratase activity. Preferably, the 3DSD polypeptide is an enzyme capable of catalyzing conversion of 3-dehydro-shikimate to protocatechuate and H₂O. A 3DSD polypeptide according to the present invention is preferably an enzyme classified under EC 4.2.1.118. For example, a suitable polypeptide having 3DSD activity includes the 3DSD polypeptide made by Podospora pauciseta, Ustilago maydis, Rhodoicoccus jostii, Acinetobacter sp., Aspergillus niger or Neurospora crassa. See, GENBANK Accession Nos. CAD60599, XP_001905369. 1, XP_761560.1, ABG93191.1, AAC37159.1, and XM 001392464. Thus, the recombinant host may include a heterologous nucleic acid encoding the 3DSD polypeptide of Podospora anserina, Ustilago maydis, Rhodoicoccus jostii, Acinetobacter sp., Aspergillus niger or Neurospora crassa or a functional homologue of any of the aforementioned sharing at least 80%, such as at least 85%, for example at least 90%, such as at least 95%, for example at least 98% sequence identity therewith.

As discussed herein, suitable wild-type OMT polypeptides are known. For example, a suitable wild-type OMT polypeptide includes the OMT made by H. sapiens, A. thaliana, or Fragaria×ananassa NM_0000754, AY062837; and AF220491 as well as Nos. OMT polypeptides isolated from a variety of other mammals, plants or microorganisms.

Likewise, suitable wild-type AROM polypeptides are known. For example, suitable wild-type AROM polypeptides include AROM made by S. cerevisiae, S. pombe, S. japonicas, N. crassa, and Y. lipolytica. See GENBANK Accession Nos. X06077, NP 594681.1, XP 002171624 and XP 956000.

Suitable ACAR polypeptides are known. An ACAR polypeptide according to the present invention may be any enzyme having aromatic carboxylic acid reductase activity. Preferably, the ACAR polypeptide is an enzyme capable of catalyzing conversion protocatechuic acid to protocatechuic aldehyde and/or conversion of vanillic acid to vanillin. An ACAR polypeptide according to the present invention is preferably an enzyme classified under EC 1.2.1.30. For example a suitable ACAR polypeptide is made by Nocardia sp. See, e.g., GENBANK Accession No. AY495697. Thus, the recombinant host may include a heterologous nucleic acid encoding the ACAR polypeptide of Nocardia sp. or a functional homologue thereof sharing at least 80%, such as at least 85%, for example at least 90%, such as at least 95%, for example at least 98% sequence identity therewith.

Suitable PPTase polypeptides are known. A PPTase polypeptide according to the present invention may be any enzyme capable of catalyzing phosphopantetheinylation. Preferably, the PPTase polypeptide is an enzyme capable of catalyzing phosphopantetheinylation of ACAR. For example, a suitable PPTase polypeptide is made by E. coli, Corynebacterium glutamicum, or Nocardia farcinica. See GENBANK Accession Nos. NP 601186, BAA35224, and YP 120266. Thus, the recombinant host may include a heterologous nucleic acid encoding the PPTase polypeptide of E. coli, C. glutamicum, or N. farcinica or a functional homologue of any of the aforementioned sharing at least 80%, such as at least 85%, for example at least 90%, such as at least 95%, for example at least 98% sequence identity therewith.

Glucosylation of vanillin is particularly useful. Vanillin-β-D-glucoside is the storage form of vanillin found in the vanilla pod. It is non-toxic to most organisms, including yeast, and has a higher solubility in water, as compared to vanillin. In addition, the formation of vanillin-β-D-glucoside most likely directs the biosynthesis toward vanillin production. UGT72E2 (Hansen, et al. (2009) Appl. Environ. Microbial. 75:2765-27740) exhibited high substrate specificity toward vanillin. observation, its expression in cerevisiae strain resulted in In concordance with this the vanillin producing S. almost all vanillin being converted into vanillin-β-D-glucoside. The ability to turn vanillin into vanillin-β-D-glucoside in vivo is important, because microbial production of non-glucosylated vanillin beyond the 0.5-1 g/liter scale would be hampered by the toxicity of free vanillin. Glucosylation serves to circumvent the inhibitory effect.

Accordingly, the recombinant host of this invention also expresses a UGT polypeptide. A UGT polypeptide may be any UDP-Glucose:Aglycon-Glucosyltransferase. Preferably the UGT polypeptides can catalyze the glucosylation of vanillin (i.e., to produce vanillin beta-D-glucoside). Thus, the UGT polypeptide may be a Family 1 glycosyltransferease. Preferred UGT polypeptides according to the invention are classified under EC 2.4.1. Suitable UGT polypeptides include the UGT71C2, UGT72B1, UGT72E2, UGT84A2, UGT89B1, UGT85B1, and arbutin synthase polypeptides. See, e.g., GENBANK Accession Nos. AC0005496, NM_116337, and NM_126067. The A. thaliana UGT72E2 is particularly useful (see, e.g., Hansen, et al. (2009) supra). Thus, the recombinant host may include a heterologous nucleic acid encoding the UGT71C2, UGT72B1, UGT72E2, UGT84A2, UGT89B1, UGT85B1, or arbutin synthase or a functional homologue of any of the aforementioned sharing at least 80%, such as at least 85%, for example at least 90%, such as at least 95%, for example at least 98% sequence identity therewith. Other useful UGTs are described in WO 01/40491.

As a further embodiment of this invention, a VAO enzyme (EC 1.1.3.38) can also be expressed by host cells to oxidize any formed vanillyl alcohol into vanillin. VAO enzymes are known in the art and include, but are not limited to enzymes from filamentous fungi such as Fusarium moniliformis (GENBANK Accession No. AFJ11909) and Penicillium simplicissium (GENBANK Accession No. P56216; Benen, et al. (1998) J. Biol. Chem. 273:7865-72) and bacteria such as Modestobacter marinus (GENBANK Accession No. YP_006366868), Rhodococcus jostii (GENBANK Accession No. YP 703243.1) and R. opacus (GENBANK Accession No. EHI39392).

In some cases, it is desirable to inhibit one or more functions of an endogenous polypeptide in order to divert metabolic intermediates toward vanillin or vanillin glucoside biosynthesis. For example, pyruvate decarboxylase (PDC1) and/or glutamate dehydrogenase activity can be reduced. In such cases, a nucleic acid that inhibits expression of the polypeptide or gene product may be included in a recombinant construct that is transformed into the strain. Alternatively, mutagenesis can be used to generate mutants in genes for which it is desired to inhibit function.

A number of prokaryotes and eukaryotes are suitable for use in constructing the recombinant microorganisms described herein, e.g., gram-negative bacteria, gram-positive bacteria, yeast or other fungi. A species and strain selected for use as a vanillin or vanillin glucoside production strain is first analyzed to determine which production genes are endogenous to the strain and which genes are not present. Genes for which an endogenous counterpart is not present in the strain are assembled in one or more recombinant constructs, which are then transformed into the strain in order to supply the missing function(s).

Exemplary prokaryotic and eukaryotic species are described in more detail below. However, it will be appreciated that other species may be suitable. For example, suitable species may be in a genus Agaricus, Aspergillus, Bacillus, Candida, Corynebacterium, Escherichia, Fusarium Gibberella, Kluyveromyces, Laetiporus, Lentinus, Phaffia, Phanerochaete, Pichia, Physcomitrella, Rhodoturula, Saccharomyces, Xanthophyllomyces Schizosaccharomyces, Sphaceloma, Yarrowia and Lactobacillus. Exemplary species from such genera include Lentinus tigrinus, Laetiporus sulphureus, Phanerochaete chrysosporium, Pichia pastoris, Physcomitrella patens, Rhodoturula glutinis 32, Rhodoturula mucilaginosa, Phaffia rhodozyma UBV-AX, Xanthophyllomyces dendrorhous, Fusarium fujikuroi/Gibberella fujikuroi, Candida utilis and Yarrowia lipolytica. In some embodiments, a microorganism can be an Ascomycete such as Gibberella fujikuroi, Kluyveromyces lactis, Schizosaccharomyces pombe, Aspergillus niger, or Saccharomyces cerevisiae. In some embodiments, a microorganism can be a prokaryote such as Escherichia coli, Rhodobacter sphaeroides, or Rhodobacter capsulatus. It will be appreciated that certain microorganisms can be used to screen and test genes of interest in a high throughput manner, while other microorganisms with desired productivity or growth characteristics can be used for large-scale production of vanillin beta-D-glucoside.

Specific non-limiting examples of useful recombinant hosts are described in WO 01/40491, as well as in Hansen et al. (2009) Appl. Environ. Microbial. 75:2765-2774 and Brochado, et al. (2010) Microbial Cell Factories 9:84, wherein the recombinant host according to this invention contains a heterologous nucleic acid encoding a mutant COMT polypeptide and/or mutant AROM polypeptide instead of the OMT genes described in WO 01/40491.

One preferred recombinant host to use with the present invention is S. cerevisiae, which may be recombinantly engineered as described herein. S. cerevisiae is a widely used chassis organism in synthetic biology, and can be used as the recombinant microorganism platform. There are libraries of mutants, plasmids, detailed computer models of metabolism and other information available for S. cerevisiae, allowing for rational design of various modules to enhance product yield. Methods are known for making recombinant microorganisms. The VG4 strain of S. cerevisiae (Brochado, et al. (2010) Microb. Cell Fact. 9:84) is particularly useful. VG4 has the genotype of pdc1Δgdhl↑tGDH2.

Aspergillus species such as A. oryzae, A. niger and A. sojae are widely used microorganisms in food production, and can also be used as the recombinant microorganism platform. Thus, the recombinant host may be Aspergillus spp. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for the production of food ingredients such as vanillin and vanillin glucoside.

E. coli, another widely used platform organism in synthetic biology, can also be used as the recombinant microorganism platform. Thus, the recombinant host may be E. coli. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

Rhodobacter can be used as the recombinant microorganism platform. Thus, the recombinant host may be Rhodobacter spp. Similar to E. coli, there are libraries of mutants available as well as suitable plasmid vectors, allowing for rational design of various modules to enhance product yield. Methods similar to those described above for E. coli can be used to make recombinant Rhodobacter microorganisms.

Physcomitrella mosses, when grown in suspension culture, have characteristics similar to yeast or other fungal cultures. This genera is becoming an important type of cell for production of plant secondary metabolites, which can be difficult to produce in other types of cells. Thus, the recombinant host may be a Physcomitrella spp.

In some embodiments, the nucleic acids and polypeptides described herein are introduced into plants or plant cells to increase overall vanillin or vanillin glucoside production. Thus, a recombinant host can be a plant or a plant cell that includes at least one heterologous nucleic acid described herein. A plant or plant cell can be transformed by having a heterologous nucleic acid integrated into its genome, i.e., can be stably transformed. Stably transformed cells typically retain the introduced nucleic acid with each cell division. A plant or plant cell can also be transiently transformed such that the heterologous nucleic acid is not integrated into its genome. Transiently transformed cells typically lose all or some portion of the introduced nucleic acid with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a heterologous nucleic acid, for example a recombinant nucleic acid construct into other lines, to transfer a heterologous nucleic acid to other species, or for further selection of other desirable traits. Alternatively, transgenic plants can be propagated vegetatively for those species amenable to such techniques. As used herein, a transgenic plant also refers to progeny of an initial transgenic plant provided the progeny inherits the transgene. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation; see U.S. Pat. Nos. 5,538,880; 5,204,253; 6,329,571; and 6,013,863. If a cell or cultured tissue is used as the recipient tissue for transformation, plants can be regenerated from transformed cultures if desired, by techniques known to those skilled in the art.

A population of transgenic plants can be screened and/or selected for those members of the population that have a trait or phenotype conferred by expression of the transgene. For example, a population of progeny of a single transformation event can be screened for those plants having a desired level of expression of a polypeptide or nucleic acid described herein. Physical and biochemical methods can be used to identify expression levels. These include Southern analysis or PCR amplification for detection of a polynucleotide; northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, western blots, immunoprecipitation, and enzyme-linked immunoassays to detect polypeptides. Other techniques such as in situ hybridization, enzyme staining, and immunostaining also can be used to detect the presence or expression of polypeptides and/or nucleic acids. Methods for performing all of the referenced techniques are known.

As an alternative, a population of plants with independent transformation events can be screened for those plants having a desired trait, such as production of vanillin glucoside. Selection and/or screening can be carried out over one or more generations, and/or in more than one geographic location. In some cases, transgenic plants can be grown and selected under conditions which induce a desired phenotype or are otherwise necessary to produce a desired phenotype in a transgenic plant. In addition, selection and/or screening can be applied during a particular developmental stage in which the phenotype is expected to be exhibited by the plant. Selection and/or screening can be carried out to choose those transgenic plants having a statistically significant difference in vanillin or vanillin beta-D-glucoside level relative to a control plant that lacks the transgene.

### Functional Homologs

Functional homologs of the polypeptides described above are also suitable for use in producing vanillin or vanillin glucoside in a recombinant host. Thus, the recombinant host may include one or more heterologous nucleic acid(s) encoding functional homologs of the polypeptides described above and/or a heterologous nucleic acid encoding a mutant COMT or AROM polypeptide as described herein. A functional homolog is a polypeptide that has sequence similarity to a reference polypeptide, and that carries out one or more of the biochemical or physiological function(s) of the reference polypeptide. A functional homolog and the reference polypeptide may be natural occurring polypeptides, and the sequence similarity may be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, may themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional AROM and/or COMT polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide: polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of AROM or COMT polypeptides, 3DSD, ACAR, PPTase, or UGT polypeptides. Sequence analysis can involve BLAST, Reciprocal BLAST, or PSI-BLAST analysis of nonredundant databases using an AROM or COMT, 3DSD, ACAR, a PPTase, or UGT amino acid sequence as the reference sequence. Amino acid sequence is, in some instances, deduced from the nucleotide sequence. Those polypeptides in the database that have greater than 40% sequence identity are candidates for further evaluation for suitability as vanillin or vanillin glucoside biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in AROM or COMT polypeptides or vanillin biosynthesis polypeptides, e.g., conserved functional domains.

Conserved regions can be identified by locating a region within the primary amino acid sequence of a polypeptide that is a repeated sequence, forms some secondary structure (e.g., helices and beta sheets), establishes positively or negatively charged domains, or represents a protein motif or domain. See, e.g., the Pfam database describing consensus sequences for a variety of protein motifs and domains. The information included at the Pfam database is described in Sonnhammer, et al. (1998) Nucl. Acids Res. 26:320-322; Sonnhammer et al. (1997) Proteins 28:405-420; and Bateman et al. (1999) Nucl. Acids Res. 27:260-262. Conserved regions also can be determined by aligning sequences of the same or related polypeptides from closely related species. Closely related species preferably are from the same family. In some embodiments, alignment of sequences from two different species is adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity. Sequence identity can be determined as set forth above.

The invention will be further described in the following examples, which do not limit the scope of the invention described in the claims.

### Example 1: Yeast Reporter Strain for Producing Vanillin Glucoside from Glucose

A genetically stable yeast strain producing vanillin glucoside from glucose was created as described in Brochado, et al. (2010) Microb. Cell Fact. 9:84, i.e., strain VG4 with a gene deletion of PDC1 (Pyruvate decarboxylase) and GDH1 (Glutamate dehydrogenase), and overexpressing GDH2. In addition, the strain harbored an expression construct containing a PPTase integrated into the ECM3 inter-locus region of the yeast genome. Expression of the Corynebacterium glutamicum PPTase coding sequence was controlled by the yeast TPI1 promoter (Hansen, et al. (2009) Appl. Environ. Microbial. 75(9):2765-74. Epub 2009 Mar. 13). The resulting strain was designated V12.

### Example 2: Construction of an AROM Lacking Domain 5

The 5′-nearest 3912 bp of the yeast ARO1 gene, which includes all functional domains except domain 5 (having the shikimate dehydrogenase activity), was isolated by PCR amplification from genomic DNA prepared from S. cerevisiae strain S288C, using proof-reading PCR polymerase. The resulting DNA fragment was sub-cloned into the pTOPO vector and sequenced to confirm the DNA sequence. The nucleic acid sequence and corresponding amino acid sequence are presented in SEQ ID NO:1 and SEQ ID NO:2, respectively. This fragment was subjected to a restriction digest with SpeI and Sal1 and cloned into the corresponding restriction sites in the high copy number yeast expression vector p426-GPD (a 2μ-based vector), from which the inserted gene can be expressed by the strong, constitutive yeast GPD1 promoter. The resulting plasmid was designated pVAN133.

### Example 3: Yeast AROM with Single Amino Acid Substitutions in Domain 5

All mutant AROM polypeptides described in this example are polypeptides of SEQ ID NO:4, wherein one amino acid has been substituted for another amino acid. The mutant AROM polypeptides are named as follows: XnnnY, where nnn indicates the position in SEQ ID NO:4 of the amino acid, which is substituted, X is the one letter code for the amino acid in position nnn in SEQ ID NO:4 and Y is the one letter code for the amino acid substituting X. By way of example A1533P refers to a mutant AROM polypeptide of SEQ ID NO:4, where the alanine at position 1533 is replaced with a proline.

The full 4764 bp ARO1 gene was isolated by PCR amplification from genomic DNA prepared from S. cerevisiae strain S288C, using proof-reading PCR polymerase. The resulting DNA fragment was sub-cloned into the pTOPO vector and sequenced to confirm the DNA sequence. The nucleic acid sequence and corresponding amino acid sequence are presented in SEQ ID NO:3 and SEQ ID NO:4, respectively. This fragment was subjected to a restriction digest with SpeI and SalI and cloned into the corresponding restriction sites in the low copy number yeast express vector p416-TEF (a CEN-ARS-based vector), from which the gene can be expressed from the strong TEF promoter. The resulting plasmid was designated pVAN183.

Plasmid pVAN183 was used to make 10 different domain 5 mutants of ARO1, using the QUICKCHANGE II Site-Directed Mutagenesis Kit (Agilent Technologies). With reference to SEQ ID NO:4, the mutants contained the following amino acid substitutions: A1533P, P1500K, R1458W, V1349G, T1366G, 11387H, W1571V, T1392K, K1370L and A1441P.

After sequence confirmation of these mutant AROM genes, the expression plasmids containing the A1533P, P1500K, R1458W, V1349G, T1366G, I1387H, W1571V, T1392K, K1370L and A1441P substitutions were designated pVAN368-pVAN377, respectively. The nucleic acid sequences and corresponding amino acid sequences of the AROM mutants are listed in Table 1.

TABLE 1

| | | Nucleic Acid | Amino Acid |
|---|---|---|---|
| Mutant | Plasmid | Sequence | Sequence |
| A1533P | pVAN368 | SEQ ID NO: 5 | SEQ ID NO: 6 |
| P1500K | pVAN369 | SEQ ID NO: 7 | SEQ ID NO: 8 |
| R1458W | pVAN370 | SEQ ID NO: 9 | SEQ ID NO: 10 |
| V1349G | pVAN371 | SEQ ID NO: 11 | SEQ ID NO: 12 |
| T1366G | pVAN372 | SEQ ID NO: 13 | SEQ ID NO: 14 |
| I1387H | pVAN373 | SEQ ID NO: 15 | SEQ ID NO: 16 |
| W1571V | pVAN374 | SEQ ID NO: 17 | SEQ ID NO: 18 |
| T1392K | pVAN375 | SEQ ID NO: 19 | SEQ ID NO: 20 |
| K1370L | pVAN376 | SEQ ID NO: 21 | SEQ ID NO: 22 |
| A1441P | pVAN377 | SEQ ID NO: 23 | SEQ ID NO: 24 |

### Example 4: Yeast AROM and 3DHS Dehydratase Fusion Protein

The 5′-nearest 3951 bp of the yeast ARO1 gene, which includes all functional domains except domain 5 with the shikimate dehydrogenase activity, was isolated by PCR amplification from genomic DNA prepared from S. cerevisiae strain S288C, using proof-reading PCR polymerase. The resulting DNA was sub-cloned into the pTOPO vector and sequenced to confirm the DNA sequence. In order to fuse this fragment to the 3-dehydroshikimate dehydratase (3DSD) gene from the vanillin pathway, the 3DSD gene from Podospora pauciseta (Hansen, et al. (2009) supra) was inserted into the XmaI-EcoRI sites of yeast expression vector p426-GPD, and then the cloned ARO1 fragment was liberated and inserted into the SpeI-XmaI sites of the resulting construct. The final fusion gene is expressed from the strong, constitutive GPD1 promoter. The resulting plasmid was named pVAN132. The nucleic acid sequence and corresponding amino acid sequence of this fusion protein are presented in SEQ ID NO:25 and SEQ ID NO:26, respectively.

### Example 5: Expression of Mutant or Fusion AROM Enzymes in Yeast Already Biosynthesizing Vanillin Glucoside

Each of the plasmids described in Examples 2, 3 and 4 were introduced into strain V12 by transformation, us the lithium acetate transformation protocol, resulting in the following yeast strains: V12-Arol-1 (containing plasmid pVAN133), V12-Arol-2 (containing plasmid pVAN132), V12-1-3 (containing plasmid pVAN183), V12-Arol-4 (containing plasmid pVAN368), V12-Arol-5 (containing plasmid pVAN369), V12-Arol-6 (containing plasmid pVAN370), V12-Arol-7 (containing plasmid pVAN371), V12-Arol-8 (containing plasmid pVAN372), V12-Arol-9 (containing plasmid pVAN373), V12-Arol-10 (containing plasmid pVAN374), V12-Arol-11 (containing plasmid pVAN375), V12-Arol-12 (containing plasmid pVAN376) and V12-Arol-13 (containing plasmid pVAN377).

Yeast strains V12-Arol-1, V12-Arol-2, V12-1-3, V12-Arol 4, V12-Arol-5, V12-Arol-6, V12-Arol-7, V12-Arol-8, V12-Arol-9, V12-Arol-10, V12-Arol-11, V12-Arol-12, and V12-Arol-13 were grown as 200 ml cultures in 500 ml Erlenmeyer shake flasks using SC (synthetic complete) growth medium without aromatic amino acids, at 30° C. with moderate revolution (150 rpm) for 72 hours. Samples were taken at 48 hours, and the content of vanillin glucoside was determined. Yeast strain V12 containing the empty vectors p416-TEF or p426-GPD were included as controls. Vanillin glucoside (VG) production in the control strains (containing empty plasmids p416- and p426-GPD) was typically around 250 mg/L. Expressing the domain 5 truncated AROM is expected to increase VG production (strain V12-Arol-1) and further physical fusion of this truncated AROM to the first committed enzyme in the heterologous vanillin pathway (Podospora pauciseta 3DSD) is expected to result in a further increase in vanillin glucoside production (strain V12-Arol-2).

Of the mutant versions of AROM, in which single amino acids of domain 5 were changed, the T1392K (strain V12-Arol-9) and K1370L (strain V12-Arol-10) may be of use for increasing VG production. For example, an approximately 30-35% increase in VG production may be observed for T1392K (strain V12-Arol-9) and K1370L (strain V12-Arol-10).

This example demonstrates that by over-expressing a mutant AROM polypeptide with decreased shikimate dehydrogenase activity, the cellular concentration of 3-DHS can be increased sufficiently to play a role in the final titer yielded in the pathway. This example also demonstrates that fusing the first enzyme of the heterologous pathway, i.e., 3DHD, to a truncated AROM enzyme results in an augmentation of flux into the heterologous pathway, obtaining substrate channeling. Finally, the experiments described herein indicated that by changing discrete amino acids in the AROM domain naturally metabolizing 3DHS, the compound needed for vanillin production, the amount of the 3DHS available for vanillin biosynthesis can be increased.

### Example 6: COMT Mutants

All mutant COMT polypeptides described in this example are polypeptides of SEQ ID NO:27, wherein one amino acid has been substituted for another amino acid. The mutant COMT polypeptides are named as follows: XnnnY, where nnn indicates the position in SEQ ID NO:27 of the amino acid, which is substituted, X is the one letter code for the amino acid in position nnn in ID NO:27 and Y is the one letter code for the amino substituting X. By way of L198Y refers to a mutant COMT polypeptide of SEQ ID NO:27, where the leucine at position 198 is replaced with a tryptophan.

Nucleic acids encoding mutant COMT polypeptides were constructed by PCR using primers containing the desired codons. PCRs were either done as a single PCR or using sequence overlap extension PCR (SOE) by standard procedures. This step may, for example, be done by commercial providers, such as Life Technologies. The primers used are listed in Table 2.

TABLE 2

| | | SEQ ID |
|---|---|---|
| Primer | Sequence | NO: |
| HSOMT_F | CGTAGCATGCAGTCTAGAAAAATGGGTGACACTAA | 28 |
| | GGAGC | |
| HSOMT_R | GACGACGTTAGTGACAgaattcTTATGGACCAGCT | 29 |
| | TCAGAACCTG | |
| HSOMT2_F | CGTAGCATGCAGTCTAGAaaaATGG | 30 |
| HSOMT2_R | GACGACGTTAGTGACAgaattc | 31 |
| W38NRK_F | CTATTGACACTTATTGTGAGCAAAAGGAGnrKGCT | 32 |
| | ATGAACGTTG | |
| W38NYK_F | CTATTGACACTTATTGTGAGCAAAAGGAGnyKGCT | 33 |
| | ATGAACGTTG | |
| W38_R | CTCCTTTTGCTCACAATAAGTGTCAATAG | 34 |
| M40NRK_F | GACACTTATTGTGAGCAAAAGGAGTGGGCTnrKAA | 35 |
| | CGTTGGTGAC | |
| M40NRK_R | CACTTATTGTGAGCAAAAGGAGTGGGCTnyKAACG | 36 |
| | TTGGTGAC | |
| M40_R | CTCCTTTTGCTCACAATAAGTGTC | 37 |
| W143NRK_F | CTTTGGACATGGTTTTCTTGGACCATnrKAAGGAC | 38 |
| | AGATATTTGCC | |
| W143NYK_F | CTTTGGACATGGTTTTCTTGGACCATnyKAAGGAC | 39 |
| | AGATATTTGCC | |
| W143_R | ATGGTCCAAGAAAACCATGTCCAAAG | 40 |
| C173NRK_F | GTACTGTTTTGTTAGCTGACAACGTTATTnrKcca | 41 |
| | GGTGCTCCAGACTTCTTG | |
| C173NYK_F | GTACTGTTTTGTTAGCTGACAACGTTATTnyKcca | 42 |
| | GGTGCTCCAGACTTCTTG | |
| C173_R | AATAACGTTGTCAGCTAACAAAACAGTAC | 43 |
| P174NRK_F | CTGTTTTGTTAGCTGACAACGTTATTtgtnrKGGT | 44 |
| | GCTCCAGACTTC | |
| P174NYK_F | CTGTTTTGTTAGCTGACAACGTTATTtgtnyKGGT | 45 |
| | GCTCCAGACTTC | |
| P174_R | ACAAATAACGTTGTCAGCTAACAAAACAG | 46 |
| L198NRK_R | atttagaattcTTATGGACCAGCTTCAGAACCTGG | 47 |
| | ACCCTTATATATAGCCTTCTCCAAACCGTCAACAA | |
| | CCTCTCTATATTCMYNGAAAGATTGATAATGAG | |
| L198NYK_R | atttagaattcTATGGACCAGCTTCAGAACCTGGA | 48 |
| | CCCTTATATATAGCCTTCTCCAAACCGTCAACAAC | |
| | CTCTCTATATTCMRNGAAAGATTGATAATGAG | |
| E199NRK_R | atttagaattcTTATGGACCAGCTTCAGAACCTGG | 49 |
| | ACCCTTATATATAGCCTTCTCCAAACCGTCAACAA | |
| | CCTCTCTATAMYNCAAGAAAGATTGATAATG | |
| E199NYK_R | atttagaattcTTATGGACCAGCTTCAGAACCTGG | 50 |
| | ACCCTTATATATAGCCTTCTCCAAACCGTCAACAA | |
| | CCTCTCTATAMRNCAAGAAAGATTGATAATG | |
| R201NRK_R | atttagaattcTTATGGACCAGCTTCAGAACCTGG | 51 |
| | ACCCTTATATATAGCCTTCTCCAAACCGTCAACAA | |
| | CCTCMYNATATTCCAAGAAAGATTG | |
| R201NYK_R | atttagaattcTTATGGACCAGCTTCAGAACCTGG | 52 |
| | ACCCTTATATATAGCCTTCTCCAAACCGTCAACAA | |
| | CCTCMRNATATTCCAAGAAAGATTG | |

In the primer sequences provided above: M may be A or C; R may be A or G; W may be A or T; S may be G or C; Y may be C or T; K may be G or T; V may be A, G or C; H may be A, C or T; D may be A, G or T; B may be G, C or T; and N may be A, G, C or T.

Restriction sites for EcoRI/XbaI were included in the primers to facilitate cloning of the PCR products into the centromeric yeast expression vector p416-TEF (Mumberg, et al. (1995) Gene 156(1):119-22). The resulting plasmids were transformed into a Yeast strain EFSC2055 (Genotype: Mata his3Dl leu2DO metl500 ura3DO adh6::LEU2 bg11::KanMX4 PTPI1::3DSD[AurC]::(HsOMT::MET15[NatMX])::ACAR[HphMX]::UGT7 2E2[HIS3]ECM3::(CorPPTase-ScHAP4). This yeast strain is based on the VG4 strain (Brochado, et al. (2010) Microbial. Cell Factories 9:84), and further includes a disruption of HsOMT with MET15 marker, and has two additional genes integrated, namely Corynebacterium glutamicum PPtase (NCBI database accession no. NP 601186) and S. cerevisiae HAP4 (NCBI database accession no. Z28109).

The HsOMT was disrupted by PCR amplifying S. cerevisiae MET15 (methionine auxotrophic selection marker) using primers with 70 bp tails homologous to the front and back end of HsOMT, respectively. The yeast strain was then transformed with the PCR product, resulting in transformants having no HsOMT activity and able to grow on plates not supplemented with methionine. The resulting strain was designated EFSC2055,

Plasmids expressing CorPPTase and ScHAP4 were transformed into EFSC2055 predecessor strain using the standard yeast transformation lithium acetate/PEG protocol and transformants were selected on SC-uracil plates. Transformants were tested by growing in 3 ml cultures for 72 hours in Delft medium supplemented with 8% sugar beet molasses.

Cultures were analyzed using HPLC-UV to quantify vanillin glucoside/Isovanillin glucoside and related products. HPLC analysis was carried out with an AGILENT 1100 series system with binary pump and a Phenomenex Synergi Polar-RP 2.5u 100 Å 100×2.00 mm column, which separates precursors and Isovanillin and vanillin. A flat gradient was run with water/acetonitrile+0.1% trifluoroacetic acid. A 8.9 minute program+1.1 minute postrun was carried out as presented in Table 3.

TABLE 3

| Time | % Acetonitrile | Flow ml/min. |
|---|---|---|
| 0 | 5 | 0.5 |
| 0.7 | 5 | 0.5 |
| 5.7 | 27 | 0.5 |
| 6.2 | 100 | 0.5 |
| 6.6 | 100 | 0.7 |
| 7.8 | 100 | 1.0 |
| 8.1 | 100 | 1.0 |
| 8.6 | 5 | 0.8 |
| 8.9 | 5 | 0.6 |

Vanillin glucoside and isovanillin glucoside were quantified by integrating the area of the HPLC peaks and comparing the same with a standard curve. The results of this analysis are shown in FIG. 3A. Yeast cells expressing the wild-type Hs-OMT of SEQ ID NO:27 (referred to as Hs-COMT wt) produced isovanillin and vanillin in a ratio of approximately 1:3, whereas the mutant L198Y produced isovanillin and vanillin in a ratio of approximately 1:125. The exact ratio was difficult to determine as isovanillin production was at the detection limit.

In addition to the mutations analyzed in FIG. 3A, good specificity and low isovanillin production in the mutants L198C, L198N, L198D, L198F, and L198E.

### Example 7: Reduction of Vanillyl Alcohol

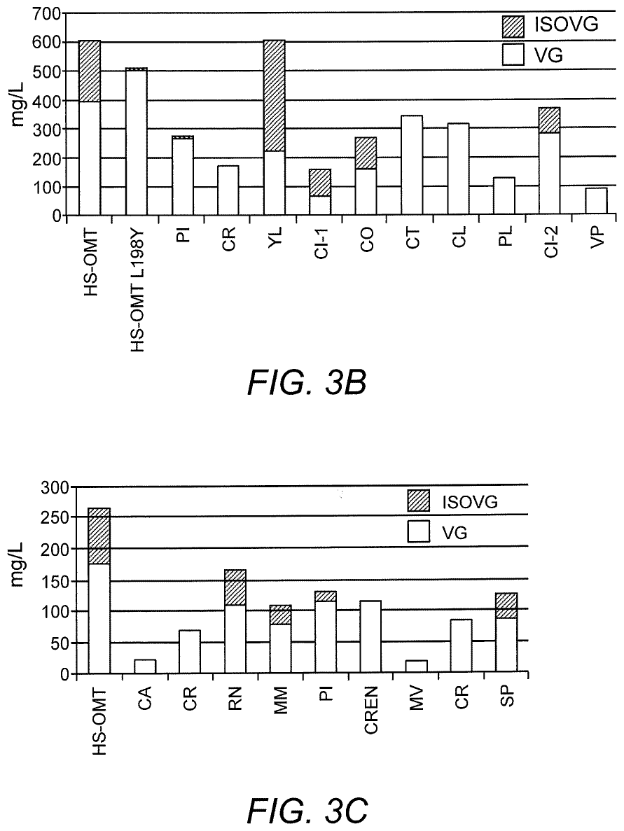

By way of illustration, P. simplicissium (GENBANK Accession No. P56216) and R. jostii (GENBANK Accession No. YP 703243.1) VAO genes were isolated and cloned into a yeast ssion vector. The expression vectors were subsequent transformed a yeast expressing glucosyltransferase. The transformed strains were tested for VAO activity by growing the yeast for 48 hours in medium supplemented with 3 mM vanillyl alcohol. The results of this analysis are presented in FIG. 4. VAO enzymes from both P. simplicissium and R. jostii exhibited activity in yeast. When the VAO enzymes were analyzed in a strain capable of producing vanillin glucoside, there was a reduction in the accumulation of llyl alcohol during vanillin glucose fermentation.

### Example 8: ACAR Gene from Neurospora arassa

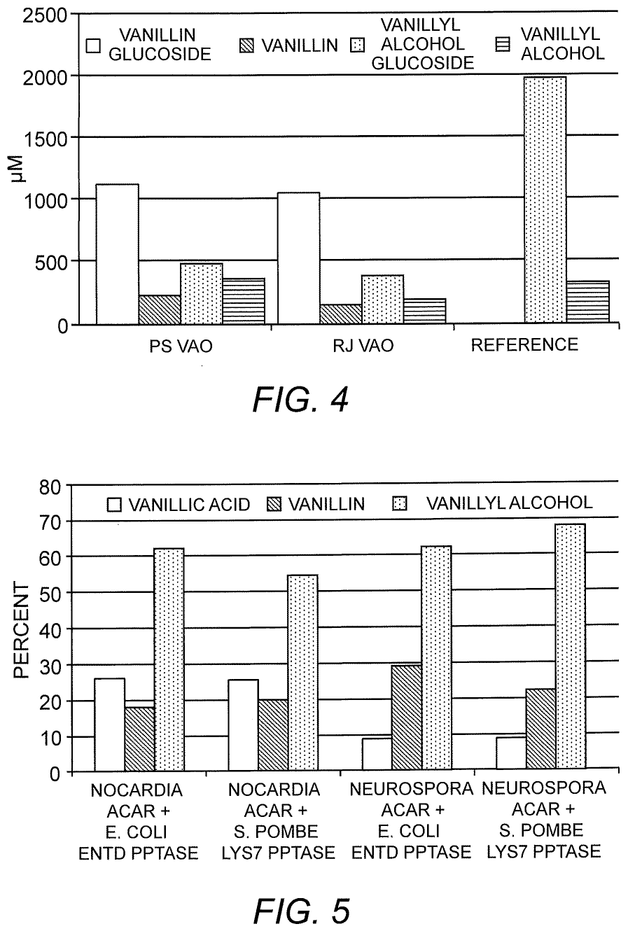

As an alternative to an ACAR protein (EC 1.2.1.30) from Nocardia iowensis (Hansen, et al. ((2009) Appl. Environ. Microbial. 75:2765-74), the use of a Neurospora crassa ACAR enzyme (Gross & Zenk (1969) Eur. J. Biochem. 8:413-9; U.S. Pat. No. 6,372,461) in yeast was investigated, as Neurospora (bread mold) is a GRAS organism. An N. crassa gene (GENBANK XP 955820) with homology to the Nocardia iowensis ACAR was isolated and cloned into a yeast expression vector. The vector was transformed into a yeast strain expressing a PPtase, strains were selected for the presence of the ACAR gene, and the selected yeast was cultured for 72 hours in medium supplemented with 3 mM vanillic acid to demonstrate ACAR activity. The results of this analysis are presented in FIG. 5. The N. crassa ACAR enzyme was found to a higher activity in yeast than the N. iowensis ACAR. Therefore, in some embodiments of the method disclosed herein, a N. crassa ACAR enzyme is used in the production of vanillin or vanillin glucoside.

In addition to N. iownsis or N. crassa ACAR proteins, it is contemplated that other ACAR proteins may be used, including but not limited to, those isolated from Nocardia liensis (GENBANK Accession No. 6728), Nocardia farcinica (GENBANK Access No. BAD56861), Podospora anserina (GENBANK Accession No. CAP62295), or Sordaria macropora (GENBANK Accession No. CCC14931), which significant sequence identity with the N. iownsis or N. crassa ACAR protein.

What is claimed is:

1. A method for producing vanillin and/or vanillin beta-D-glucoside comprising
(a) providing a recombinant host capable of producing vanillin, wherein said recombinant host harbors a heterologous nucleic acid encoding Rhodococcus jostii, Rhodococcus opacus or Modestobacter marinus vanillyl alcohol oxidase; and
(b) cultivating said recombinant host for a time sufficient for said recombinant host to produce vanillin and/or vanillin glucoside, wherein said recombinant host exhibits a reduction in the accumulation of vanillyl alcohol.

2. The method of claim 1, wherein said host is a microorganism.

3. The method of claim 2, wherein said host is Saccharomyces cerevisiae, Schizosaccharomyces pombe or Escherichia coli.

4. The method of claim 1, wherein said host is a plant or plant cell.

5. The method of claim 1, wherein said host further comprises a gene encoding a 3-dehydroshikimate dehydratase (3DSD), a gene encoding a aromatic carboxylic acid reductase (ACAR), a gene encoding a uridine 5′-diphosphoglucosyl transferase (UGT), a gene encoding a phosphopantetheine transferase (PPTase) and/or a gene encoding a catechol-O-methyl transferase (COMT).

6. An extract comprising vanillin and/or vanillin glucoside produced by the method of claim 1.

7. A consumable comprising the extract of claim 6.

8. The consumable of claim 7, wherein the consumable is a food product, pharmaceutical composition, a dietary supplement, a nutraceutical, a dental hygienic composition, a tabletop sweetener, or a cosmetic product.

9. A recombinant host comprising a heterologous nucleic acid encoding Rhodococcus jostii, Rhodococcus opacus or Modestobacter marinus vanillyl alcohol oxidase.

10. The recombinant host of claim 9, wherein the recombinant host further comprises a nucleic acid encoding a 3-dehydroshikimate dehydratase (3DSD) polypeptide, a nucleic acid encoding an aromatic carboxylic acid reductase (ACAR) polypeptide, a nucleic acid encoding a phosphopantetheine transferase (PPTase) polypeptide, a nucleic acid encoding an uridine 5′-diphosphoglucosyl transferase (UGT) polypeptide and/or a nucleic acid encoding catechol-O-methyl transferase (COMT).

11. The recombinant host of claim 9, wherein the recombinant host is a microorganism.

12. The recombinant host of claim 11, wherein the recombinant host is Saccharomyces cerevisiae, Schizosaccharomyces pombe or Escherichia coli.

13. The recombinant host of claim 9, wherein said host is a plant or plant cell.
